(19) United States
(12) Patent Application Publication
(10) Pub. No.: US 2007/0104778 A1
(43) Pub. Date: May 10, 2007

(54) Controlled-release emulsion compositions

(76) Inventors: Hongxia Zeng, Newtown, CT (US); Antonio Moroni, Morris Plains, NJ (US); Anand R. Baichwal, Wappingers Falls, NY (US); Philip A. Goliber, Brookfield, CT (US); Sara Ketsela, Danbury, CT (US); Daniel P. McNamara, Waterbury, CT (US)

Correspondence Address: DAVIDSON, DAVIDSON & KAPPEL, LLC, 485 SEVENTH AVENUE, 14TH FLOOR, NEW YORK, NY, 10018, US

(21) Appl. No.: 11/594,329

(22) Filed: Nov. 7, 2006

Related U.S. Application Data
(60) Provisional application No. 60/734,198, filed on Nov. 7, 2005.

(51) Int. Cl. A61K 36/47; A61K 31/55; A61K 9/22; A61K 9/48; A61K 31/455; A61K 31/403

(52) U.S. Cl. 424/451; 424/468; 514/217; 514/355; 514/411; 424/731

(57) ABSTRACT

The present invention is directed to controlled-release composition containing a solubilized material comprising an active agent and at least one oil-based surfactant capable of solubilizing the active agent, the solubilized material dispersed in a controlled-release particulate matrix.

This application claims the benefit of U.S. Provisional Patent Application No. 60/734,198, filed Nov. 7, 2005, the disclosure of which is hereby incorporated by reference.

### FIELD OF THE INVENTION

The present invention is directed to a controlled-release emulsion composition comprising an active agent dispersed in an emulsion and a controlled-release carrier, wherein the active agent emulsion is combined together with the controlled-release carrier to form a controlled-release composition.

### BACKGROUND OF THE INVENTION

Approximately one-third of the drugs in the United States Pharmacopoeia are water-insoluble or poorly water-soluble. Oral formulations of water-insoluble drugs or compounds with biological uses frequently show poor and erratic bioavailability. In addition, water-solubility problems delay or completely block the development of many new drugs and other biologically useful compounds.

While attempts have been made to provide pharmaceutical compositions for delivering insoluble drugs, there still exists a need in the art for improved compositions for both soluble and insoluble drugs. One method for providing compositions for soluble and insoluble drugs contemplates the use of Applicants own controlled-release technology.

U.S. Pat. Nos. 6,093,420; 6,056,977; 5,472,711; 5,455,046; 5,773,025; 5,399,358; 4,944,276; 5,128,143; 5,135,757; 5,169,639; 5,478,574; 5,399,359; 5,399,362 are directed to Applicants TIMERx® controlled-release technology. The TIMERx® technology is based on a customized, agglomerated hydrophilic complex that forms a controlled-release matrix upon compression. The matrix consists of two polysaccharides, xanthan and locust bean gum. Interactions between these components in an aqueous environment form a tight gel with a slowly-eroding core.

Another method for providing compositions for soluble and insoluble drugs contemplates the use of emulsion technology, whereby the drug is incorporated into an emulsion.

An emulsion is a dispersion containing at least two immiscible phases, a dispersed phase (the “internal phase”) having particles of dissolved and/or undissolved drug and a dispersing phase or medium (the “external or continuous phase”) in which the dispersion is immiscible. Emulsions having an oleaginous internal phase and an aqueous external phase are “oil-in-water” emulsions (o/w). Emulsions having an aqueous internal phase and an oleaginous external phase are “water-in-oil” emulsions (w/o). Emulsions have a continuous external phase that allows for there further dilution.

Emulsions may be in the form of a macro-emulsion (particle size from about μm to about 200 μm, a mini-emulsion (particle size from about 0.25 μm to about 1.0 μm or a microemulsion (particle size from about 1 nm to about 250 nm). While macro and mini-emulsions are only kinetically stable, microemulsions are thermodynamically stable, isotropic clear droplets. A microemulsion can be spontaneously formed by mixing a surfactant/co-surfactant (co-solvent) aqueous phase with an oil phase while addition of external energy is necessary to form macro and mini emulsions. With a microemulsion, drug molecules can be solubilized in the internal phase to form an oil-in-water microemulsion, or solubilized in the palisade layer of the aggregated clusters, thus giving a much higher solubilization capacity.

Emulsion preparations can be in various forms. For example, emulsions can be in a liquid or semi-solid state depending on their viscosity. Liquid emulsions are generally suitable for oral, topical, or parenteral administration, whereas semi-solid emulsions are usually administered only via the topical route (See: Ansel, Howard C., Introduction to Pharmaceutical Compositions, Fourth Edit., pp. 222-230). One specific technique utilized for providing emulsions for drugs involves the use of vitamin E and derivatives thereof. For example, U.S. Pat. No. 5,952,004 to Shire Laboratories describes a pharmaceutical composition comprising a pharmaceutical agent incorporated into a carrier emulsion comprising a hydrophobic material, e.g., d-alpha tocopherol polyethylene glycol 1000 succinate (Vitamin E TPGS) emulsified with a hydrophilic material, the drug emulsion composition being filled into soft or hard gelatin capsules, tablets or other oral compositions.

U.S. Pat. No. 6,458,373 to Sonus Pharmaceuticals, Inc., describes a pharmaceutical composition comprising α-tocopherol, a surfactant(s), e.g., vitamin E TPGS, and a therapeutic agent, wherein the composition is in the form of an emulsion. In addition, U.S. Pat. No. 6,193,895 to A/S Dumex (Dumex Ltd.) describes compositions comprising tocopherols or derivatives thereof as a solvent and/or emulsifier for insoluble drugs. In certain embodiments Vitamin E TPGS is also utilized as an emulsifier in the compositions described therein.

It would be advantageous to provide compositions that provide for increased solubility of an active agent while preferably providing increased bioavailability and stability. It would be further advantageous to provide such compositions that are suitable for use with a controlled release carrier to provide for a controlled-release of the active agent for, e.g., over a period of 12 to 24 hours.

### OBJECTS AND SUMMARY OF THE INVENTION

It is an object of the present invention to provide a composition that increases the solubility of the active agent.

It is an object of certain embodiments of the present invention to provide controlled release compositions that increase the solubility of the active agent.

It is another object of certain embodiments of the present invention to provide controlled-release compositions that provide increased bioavailability and/or stability of the active agent contained therein.

It is another object of the invention to provide a controlled-release composition of an active agent dispersed in an emulsion.

It is a further object of the present invention to provide methods for preparing such controlled-release compositions.

In accordance with the above objects and others, in certain embodiments of the present invention, there is provided a controlled-release composition that comprises an active agent dispersed in an emulsion, the emulsion containing at least one oil-based surfactant and an optional solubilizer and/or co-surfactant; and a controlled release carrier.

In certain other embodiments, there is provided a composition comprising a solubilized material comprising an active agent and at least one oil-based surfactant capable of solubilizing the active agent, the solubilized material dispersed in a controlled-release particulate matrix.

In certain other embodiments, there is provided a composition comprising a solubilized material comprising an active agent and at least one oil-based surfactant capable of solubilizing the active agent, the solubilized material coated onto a particulate controlled-release carrier.

The oil-based surfactant may be a tocopherol, derivative and/or mixture thereof. In certain embodiments, the oil-based surfactant may be D-α-tocopherol polyethylene glycol 1000 succinate (Vitamin E TPGS).

In certain embodiments, the controlled-release carrier may comprise at least one pharmaceutically acceptable gelling agent. The gelling agent may comprise at least one natural or synthetic gum. In certain preferred embodiments, the gelling agent may comprise a heteropolysaccharide gum, a homopolysaccharide gum, or a combination thereof. Preferably in combination, the homopolysaccharide gum is capable of cross-linking the heteropolysaccharide gum when exposed to an environmental fluid.

In certain other embodiments, the controlled-release carrier may comprise a controlled-release material in addition to or in place of the gelling agent.

In certain other embodiments, there is provided a controlled-release composition comprising an insoluble therapeutically active agent having a solubility of less than 1 part active agent to 30 parts water dispersed within an emulsion, wherein the emulsion contains at least one oil-based surfactant and an optional solubilizer and/or co-surfactant. The controlled-release composition of the present invention may provide release of the active agent over a period of time from about 12 to about 24 hours.

In certain embodiments, the controlled-release carrier may comprise at least one gelling agent and an optional inert diluent.

In certain embodiments, the controlled-release carrier may include a heteropolysaccharide gum and a homopolysaccharide gum in a weight ratio, e.g., of about 1:20 to about 20:1. In certain embodiments, the heteropolysaccharide gum may comprise from about 1 to about 99% by weight of the controlled-release carrier. In certain embodiments, the homopolysaccharide gum may comprise from about 1 to about 99% by weight of the controlled-release carrier.

In certain embodiments the controlled-release carrier may further contain from about 1% to about 35% by weight of an inert diluent selected from, e.g., a monosaccharide, a disaccharide, a polyhydric alcohol, or mixtures thereof.

In certain other embodiments, the controlled-release carrier may further comprise from about 1% to about 20% of an ionizable gel strength enhancing agent. Preferably the ionizable gel strength enhancing agent is included in the controlled-release carrier.

The total combined weight of the heteropolysaccharide and the homopolysaccharide may be, e.g., from about 65% to about 99% of the controlled-release carrier.

In certain embodiments, the controlled-release carrier may be incorporated into a matrix comprising the controlled-release carrier and the solubilized material, which matrix provides for the controlled-release of the active agent or a pharmaceutically acceptable salt thereof when exposed to an environmental fluid.

In certain embodiments, the controlled-release carrier may be a controlled-release coating which is coated over emulsion droplets, wherein the controlled-release coating provides for the controlled-release of the active agent or pharmaceutically acceptable salt thereof when exposed to an environmental fluid.

In certain other embodiments, the compositions of the present invention may comprise an immediate release component of active agent in addition to the controlled-release component. In certain embodiments, the composition is a bi-layered tablet, wherein one layer provides for the immediate release of the active agent and the other layer provides for the controlled-release of the active agent from the composition upon exposure to environmental fluid.

The controlled-release composition of the present invention may comprise from about 0.1% to about 10% active agent, from about 10% to about 50% emulsion and from about 40% to about 90% controlled-release carrier.

In other embodiments, the compositions may contain an optional pharmaceutically acceptable excipient that provides for an increase in the dissolution of the composition upon contact with an environmental fluid, the ratio of controlled-release carrier to pharmaceutically acceptable excipient can be, e.g., from about 60:20 to about 20:60.

In certain embodiments of the invention, a controlled-release composition is provided comprising a solubilized material comprising nifedipine or a pharmaceutically acceptable salt thereof and at least one oil-based surfactant; and a controlled-release carrier. In certain embodiments, the controlled-release carrier comprises at least one pharmaceutically acceptable gelling agent or a mixture of gelling agents, such as, but not limited to, a mixture of a heteropolysaccharide gum and a homopolysaccharide gum capable of cross-linking the heteropolysaccharide gum when the composition is exposed to an environmental fluid, and an optional pharmaceutically acceptable inert diluent.

In certain embodiments of the invention, a controlled-release composition is provided comprising a solubilized material comprising carvedilol or a pharmaceutically acceptable salt thereof and at least one oil-based surfactant; and a controlled-release carrier. In certain embodiments, the controlled-release carrier comprises at least one pharmaceutically acceptable gelling agent or a mixture of gelling agents, such as, but not limited to, a mixture of a heteropolysaccharide gum and a homopolysaccharide gum capable of cross-linking the heteropolysaccharide gum when the composition is exposed to an environmental fluid, and an optional pharmaceutically acceptable inert diluent.

In certain embodiments of the invention, a controlled-release composition is provided comprising a solubilized material comprising nimodipine or a pharmaceutically acceptable salt thereof and at least one oil-based surfactant; and a controlled-release carrier. In certain embodiments, the controlled-release carrier comprises at least one pharmaceutically acceptable gelling agent or a mixture of gelling agents, such as, but not limited to, a mixture of a heteropolysaccharide gum and a homopolysaccharide gum capable of cross-linking the heteropolysaccharide gum when the composition is exposed to an environmental fluid, and an optional pharmaceutically acceptable inert diluent.

In certain embodiments of the invention, a controlled-release composition is provided comprising a solubilized material comprising clozapine or a pharmaceutically acceptable salt thereof and at least one oil-based surfactant; and a controlled-release carrier. In certain embodiments, the controlled-release carrier comprises at least one pharmaceutically acceptable gelling agent or a mixture of gelling agents, such as, but not limited to, a mixture of a heteropolysaccharide gum and a homopolysaccharide gum capable of cross-linking the heteropolysaccharide gum when the composition is exposed to an environmental fluid, and an optional pharmaceutically acceptable inert diluent.

In certain embodiments of the invention, a controlled-release composition is provided comprising a solubilized material comprising oxcarbazepine or a pharmaceutically acceptable salt thereof and at least one oil-based surfactant; and a controlled-release carrier. In certain embodiments, the controlled-release carrier comprises at least one pharmaceutically acceptable gelling agent or a mixture of gelling agents, such as, but not limited to, a mixture of a heteropolysaccharide gum and a homopolysaccharide gum capable of cross-linking the heteropolysaccharide gum when the composition is exposed to an environmental fluid, and an optional pharmaceutically acceptable inert diluent.

In yet another embodiment of the present invention, the present invention is directed to a controlled release composition, the controlled composition comprising a transdermal delivery system comprising a solubilized material comprising an active agent and at least one oil-based surfactant capable of solubilizing the active agent, and an optional controlled release material. When the transdermal delivery system contains a controlled release material, the solubilized material may be dispersed in the controlled release material.

In yet another embodiment of the present invention, the controlled release dosage form may comprise an oral mucosal delivery system, a composition for intranasal administration, or an injectable composition.

In certain other embodiments, the invention is directed to a method of preparing a controlled release composition.

In certain embodiments, the composition may be prepared by dissolving the active agent in a suitable oil-based surfactant and/or solubilizer and adding water to obtain an emulsion; granulating the emulsion together with a controlled-release carrier to form a granulation; and incorporating the granulation into an oral solid composition.

In certain other embodiments, a transdermal delivery system may be prepared by dissolving the active agent in a suitable oil-based surfactant and/or solubilizer and adding water to obtain an emulsion and incorporating the emulsion into a transdermal delivery system.

The transdermal delivery system of the present invention may also be prepared by dissolving the active agent in a suitable oil-based surfactant and/or solubilizer and adding water to obtain an emulsion; mixing the emulsion together with a controlled-release carrier to form a mixture; and incorporating the mixture into a transdermal delivery system.

In certain embodiments, the present invention is directed to a method of treating vasosapatic angina, chronic stable angina, and/or hypertension comprising administering to a subject in need thereof a composition of the present invention. The composition may comprise a solubilized material comprising a therapeutically effective amount of nifedipine or pharmaceutically acceptable salt thereof and at least one oil-based surfactant and an optional solubilizer and/or co-surfactant; and a controlled-release carrier to form a controlled-release composition that provides release of the active agent over a period of time from about 12 to about 24 hours.

In certain embodiments, the present invention is directed to a method of treating mild or moderate heart failure and/or hypertension comprising administering to a subject in need thereof a composition of the present invention. The composition can comprise a solubilized material comprising therapeutically effective amount of carvedilol or pharmaceutically acceptable salt thereof and at least one oil-based surfactant and an optional solubilizer and/or co-surfactant; and a controlled-release carrier to form a controlled-release composition that provides release of the active agent over a period of time from about 12 to about 24 hours.

In certain embodiments, the present invention is directed to a method of reducing the incidence and severity of ischemic deficits in patients with subarachnoid hemorrhage from ruptured congenital aneurysms comprising administering to a subject in need thereof a composition of the present invention. The composition can comprise a solubilized material comprising a therapeutically effective amount of nimodipine or pharmaceutically acceptable salt thereof and at least one oil-based surfactant and an optional solubilizer and/or co-surfactant; and a controlled-release carrier to form a controlled-release composition that provides release of the active agent over a period of time from about 12 to about 24 hours.

In certain embodiments, the present invention is directed to a method of managing schizophrenia comprising administering to a subject in need thereof a composition of the present invention. The composition can comprise a solubilized material comprising a therapeutically effective amount of clozapine or pharmaceutically acceptable salt thereof and at least one oil-based surfactant and an optional solubilizer and/or co-surfactant; and a controlled-release carrier to form a controlled-release composition that provides release of the active agent over a period of time from about 12 to about 24 hours.

In certain embodiments, the invention is directed to a method of treating partial seizures comprising administering to a subject in need thereof a composition of the present invention. The composition can comprise a solubilized material comprising a therapeutically effective amount of oxcarbazepine or pharmaceutically acceptable salt thereof and at least one oil-based surfactant and an optional solubilizer or co-surfactant; and a controlled-release carrier to form a controlled-release composition that provides release of the active agent over a period of time from about 12 to about 24 hours.

In order that the invention described herein may be more fully understood, the following definitions are provided for the purpose of the disclosure:

For purposes of the present invention, “emulsion” is meant to include macro-emulsions having a particle size from about μm to about 200 μm, mini-emulsions having a particle size from about 0.25 μm to about 1.0 μm and microemulsions having a particle size from about 1 nm to about 250 nm.

By “controlled-release” it is meant for purposes of the present invention that the therapeutically active agent is released from the formulation at a controlled rate such that therapeutically beneficial blood levels (but below toxic levels) of the medicament are maintained over an extended period of time, e.g., providing a 12 hour or a 24 hour composition.

By “bioavailable” it is meant for purposes of the present invention that the therapeutically active agent is absorbed from the controlled-release composition and becomes available in the body at the intended site of drug action.

The term “environmental fluid” is meant for purposes of the present invention to encompass, e.g., an aqueous solution such as in-vitro dissolution media, or gastrointestinal fluid.

By “insoluble drug” is meant any therapeutic agent having a greater solubility in organics than in water. More specifically, such drugs have a water solubility of less than 10 μm/l or no greater than 1 part drug to 30 to 100 parts water (“sparingly soluble”), no greater than 1 part drug to 100 to 1,000 parts water (“slightly soluble”), no greater than 1 part drug to 1,000 to 10,000 parts water (“very slightly soluble”), or no greater than 1 part drug to 10,000 and over parts water (“insoluble”).

The term “compatible with the active agent” means for purposes of the present invention that the oil-based surfactants described herein do not destroy activity, e.g., pharmacological, or structure and that combinations of an active agent and oil-based surfactant allow for formation of the final desired product.

### BRIEF DESCRIPTION OF THE DRAWINGS

FIG. 1 shows the mean plasma concentration of clozapine for the TIMERx based Vitamin E-TPGS/N-methyl-pyrrolidinone solubilization system compared to Clozaril.

### DETAILED DESCRIPTION OF THE PREFERRED EMBODIMENTS

The invention will be described with reference to various specific and preferred embodiments and techniques, however, it should be understood that many variations and modifications can be made while remaining with the spirit and scope of the invention.

In certain embodiments of the present invention, there is provided a controlled-release composition for administering an active agent, e.g., insoluble active agent, to a patient in need thereof, such that the composition provides controlled-release of the active agent for a period from about 12 to about 24 hours. The compositions of the present invention may be prepared utilizing an emulsion of the active agent.

The emulsions utilized in the present invention can be an oil-in-water (o/w) emulsion or a water-in-oil (w/o) emulsion. Oil-in-water emulsions are achieved when the oil phase contains up to about 50% to about 70% lipids. When the emulsion is of the oil-in-water type, it is desirable that the droplet size is as small as possible. In certain embodiments an emulsion is formed having particle size in the range of from about 1 μm to about 200 μm. In certain other embodiments a microemulsion is formed having a particle size of from about 1 nm to about 250 nm. In certain preferred embodiments, a microemulsion is formed with an initial particle size in the range from about 7 nm to about 20 nm, more preferably from about or from about 9.5 nm to about 16 nm.

The emulsions utilized in the present invention are preferably alcohol free, but in certain embodiments may contain small quantities of alcohol when necessary. The emulsions can be mixed together with a controlled-release carrier to form a controlled-release composition for oral administration.

In certain embodiments of the present invention, the controlled-release composition may comprise a solubilized material comprising an active agent and at least one oil-based surfactant; and a controlled release particulate carrier.

In other embodiments, the controlled-release composition may comprise a solubilized material comprising an active agent and at least one oil-based surfactant, an optional solubilizer and/or co-surfactant; and a controlled release particulate carrier.

In certain other embodiments, the controlled-release carrier may comprise at least one pharmaceutically acceptable gelling agent and an optional inert diluent. In other embodiments, the controlled-release carrier may comprise a mixture of two or more gelling agents and an optional inert diluent.

Active Agents

The present invention contemplates the use of any and all pharmaceutically active agents, such that the composition may contain soluble to insoluble active agents. In certain preferred embodiments, the active agent is an insoluble active agent. The insoluble active agents of the present invention may have a water solubility of less than 10 gm/l or no greater than 1 part drug to 30 to 100 parts water (“sparingly soluble”). In other embodiments, the active agent may have a water solubility no greater than 1 part drug to 100 to 1,000 parts water (“slightly soluble”). In another embodiment, the active may have a solubility in water no greater than 1 part drug to 1,000 to 10,000 parts water (“very slightly soluble”). In yet other embodiments, the active may have a water solubility no greater than 1 part drug to 10,000 and over parts water (“insoluble”).

Active agents suitable for use in the present invention include, but are not limited to the free base, salts, metabolites, derivatives and any mixtures of the following : a) analgesics and anti-inflammatory agents: aloxiprin, auranofin, azapropazone, benorylate, diflunisal, etodolac, fenbufen, fenoprofen calcim, flurbiprofen, ibuprofen, indomethacin, ketoprofen, meclofenamic acid, mefenamic acid, nabumetone, naproxen, oxyphenbutazone, phenylbutazone, piroxicam, sulindac;
b) anthelmintics: albendazole, bephenium hydroxynaphthoate, cambendazole, dichlorophen, ivermectin, mebendazole, oxamniquine, oxfendazole, oxantel embonate, praziquantel, pyrantel embonate, thiabendazole;
c) anti-arrhythmic agents: amiodarone HCl, disopyramide, flecainide acetate, quinidine sulphate. Anti-bacterial agents: benethamine penicillin, cinoxacin, ciprofloxacin HCl, clarithromycin, clofazimine, cloxacillin, demeclocycline, doxycycline, erythromycin, ethionamide, imipenem, nalidixic acid, nitrofurantoin, rifampicin, spiramycin, sulphabenzamide, sulphadoxine, sulphamerazine, sulphacetamide, sulphadiazine, sulphafurazole, sulphamethoxazole, sulphapyridine, tetracycline, trimethoprim;
d) anti-coagulants: coumadin, dicoumarol, dipyridamole, nicoumalone, phenindione;
e) anti-depressants: amoxapine, maprotiline HCl, mianserin HCL, nortriptyline HCl, trazodone HCL, trimipramine maleate;
f) anti-diabetics: acetohexamide, chlorpropamide, glibenclamide, gliclazide, glipizide, glyburide, tolazamide, tolbutamide;
g) anti-epileptics: beclamide, carbamazepine, clonazepam, ethotoin, methoin, methsuximide, methylphenobarbitone, oxcarbazepine, paramethadione, phenacemide, phenobarbitone, phenytoin, phensuximide, primidone, sulthiame, valproic acid;
h) anti-fungal agents: amphotericin, butoconazole nitrate, clotrimazole, econazole nitrate, fluconazole, flucytosine, griseofulvin, itraconazole, ketoconazole, miconazole, natamycin, nystatin, sulconazole nitrate, terbinafine HCl, terconazole, tioconazole, undecenoic acid;
i) anti-gout agents: allopurinol, colchicines, probenecid, sulphin-pyrazone;
j) anti-hypertensive agents: amlodipine, benidipine, darodipine, dilitazem HCl, diazoxide, felodipine, guanabenz acetate, isradipine, minoxidil, nicardipine HCl, nifedipine, nimodipine, phenoxybenzamine HCl, prazosin HCL, reserpine, terazosin HCL;
k) anti-malarials: amodiaquine, chloroquine, chlorproguanil HCl, halofantrine HCl, mefloquine HCl, proguanil HCl, pyrimethamine, quinine sulphate;
l) anti-migraine agents: dihydroergotamine mesylate, ergotamine tartrate, methysergide maleate, pizotifen maleate, sumatriptan succinate;
m) anti-muscarinic agents: atropine, benzhexol HCl, biperiden, ethopropazine HCl, hyoscyamine, mepenzolate bromide, oxyphencylcimine HCl, tropicamide;
n) anti-neoplastic agents and immunosuppressants: aminoglutethimide, amsacrine, azathioprine, busulphan, chlorambucil, cyclosporin, dacarbazine, estramustine, etoposide, lomustine, melphalan, mercaptopurine, methotrexate, mitomycin, mitotane, mitozantrone, procarbazine HCl, tamoxifen citrate, testolactone;
o) anti-protazoal agents: benznidazole, clioquinol, decoquinate, diiodohydroxyquinoline, diloxanide furoate, dinitolmide, furzolidone, metronidazole, nimorazole, nitrofurazone, ornidazole, tinidazole;
p) anti-thyroid agents: carbimazole, propylthiouracil;
q) anxiolytic, sedatives, hypnotics and neuroleptics: alprazolam, amylobarbitone, barbitone, bentazepam, bromazepam, bromperidol, brotizolam, butobarbitone, carbromal, chlordiazepoxide, chlormethiazole, chlorpromazine, clobazam, clotiazepam, clozapine, diazepam, droperidol, ethinamate, flunanisone, flunitrazepam, fluopromazine, flupenthixol decanoate, fluphenazine decanoate, flurazepam, haloperidol, lorazepam, lormetazepam, medazepam, meprobamate, methaqualone, midazolam, nitrazepam, oxazepam, pentobarbitone, perphenazine pimozide, prochlorperazine, sulpiride, temazepam, thioridazine, triazolam, zopiclone;
r) β-Blockers: acebutolol, alprenolol, atenolol, carvedilol, labetalol, metoprolol, nadolol, nebivolol, oxprenolol, pindolol, propranolol;
s) cardiac inotropic agents: amrinone, digitoxin, digoxin, enoximone, lanatoside C, medigoxin;
t) corticosteroids: beclomethasone, betamethasone, budesonide, cortisone acetate, desoxymethasone, dexamethasone, fludrocortisone acetate, flunisolide, flucortolone, fluticasone propionate, hydrocortisone, methylprednisolone, prednisolone, prednisone, triamcinolone;
u) diuretics: acetazolamide, amiloride, bendrofluazide, bumetanide, chlorothiazide, chlorthalidone, ethacrynic acid, frusemide, metolazone, spironolactone, triamterene;
v) anti-parkinsonian agents: amantadine, apomorphine, bensazeride, benztropine mesylate, biperiden, bromocriptine mesylate, budipine, carbidopa, galantamine, levodopa, lysuride maleate, memantine, pergolide, pramipaxole, procyclidine, rivostigmine, ropinirole, scopolamine, selegeline, tacrine;
w) gastro-intestinal agents: bisacodyl, cimetidine, cisapride, diphenoxylate HCl, domperidone, famotidine, loperamide, mesalazine, nizatidine, omeprazole, ondansetron HCL, ranitidine HCl, sulphasalazine;
x) histamine H-Receptor antagonists: acrivastine, astemizole, cinnarizine, cyclizine, cyproheptadine HCl, dimenhydrinate, flunarizine HCl, loratadine, meclozine HCl, oxatomide, terfenadine;
y) lipid regulating agents: atorvastatin, bezafibrate, cerivastatin, clofibrate, fenofibrate, fluvastatin, gemfibrozil, lovastatin, pravastatin, probucol, simvastatin;
z) nitrates and other anti-anginal agents: amyl nitrate, glyceryl trinitrate, isosorbide dinitrate, isosorbide mononitrate, pentaerythritol tetranitrate;
aa) nutritional agents: betacarotene, vitamin A, vitamin B₂, vitamin D, vitamin E, vitamin K;
ab) opioid analgesics: codeine, dextropropyoxyphene, diamorphine, dihydrocodeine, meptazinol, methadone, morphine, nalbuphine, pentazocine;
ac) sex hormones: clomiphene citrate, danazol, ethinyl estradiol, medroxyprogesterone acetate, mestranol, methyltestosterone, norethisterone, norgestrel, estradiol, conjugated oestrogens, progesterone, stanozolol, stibestrol, testosterone, tibolone;
ad) stimulants: amphetamine, dexamphetamine, dexfenfluramine, fenfluramine, mazindol; and any combination or mixtures of the foregoing.

In addition to the active agents, listed above, other active agents useful in the present invention include those listed in U.S Pharmacopeia 28/National Formulary 23 (2004) and the Merck Index: An Encyclopedia of Chemicals, Drugs and Biologicals, 13^{th} Ed. (2001) the disclosure of which is hereby incorporated by reference.

In certain embodiments, the composition may contain an insoluble drug such as carvedilol, clozapine, nifedipine, nimpodipine or oxcarbazepine.

Surfactants

When the compositions of the present invention utilize an insoluble active agent and the emulsion contains only an oil-based surfactant, the oil-based surfactant must be one in which the insoluble active agent is soluble.

Oil-based surfactants suitable for use in the present invention include, but are not limited to, tocopherols, phospholipids, polysorbates, sorbitan esters of fatty acids, cetearyl glucoside, poloxamers, derivatives thereof and mixtures thereof. For example, tocopherol esters may be used according to the present invention. Thus α-tocopherol can be used as such or in the form of its esters such as α-tocopherol acetate, linoleate, nicotinate, phosphate or hemi succinate-ester, many of which are available commercially.

As used herein, the term “tocopherol” includes all such natural and synthetic tocopherol or Vitamin E compounds.

Tocopherols are a range of natural and synthetic compounds, also known by the generic term Vitamin E α-Tocopherol (chemical name: 2,5,7,8-tetramethyl-2-(4′,8′,12′-trimethyldecyl)-6-chromanole). Other members of the class include beta, gamma, and delta tocopherols, but these are not used in pure form in therapeutics, although they are present in foodstuffs. Tocopherols occur in a number of isomeric forms, the D and DL forms being most widely available.

Tocopherols or their derivatives are not typical lipid oils. They have a higher polarity than most lipids and are not soluble in water or saponifiable (the hydrolysis of a fat by an alkali with the formation of a soap and glycerol)

The melting point of natural a-tocopherol is between 2.5 and 3.5° C. Alpha-tocopherol is a viscous oil at room temperature, is soluble in most organic solvents, but insoluble in water. Although tocopherols are available naturally in foodstuffs and may be extracted from plants, α-tocopherol is now mainly produced synthetically.

In certain other preferred embodiments, the preferred oil-based surfactant is Vitamin E TPGS. Ester and ether linkages of various chemical moieties are included within the definition of vitamin E TPGS. Vitamin E TPGS is a water soluble derivative of vitamin E and consists of α-tocopherol, which is esterified with succinic acid, the other acidic group of the latter being esterified with polyethylene glycol (PEG 200-6000), preferably polyethylene glycol 1000. Vitamin E TPGS is an almost odorless waxy amphiphilic substance with a molecular weight about 1513. The melting point is about 36° C. and its solubility in water is about 20%.

In addition to the oil-based surfactants, additional surfactants suitable for use as a co-surfactant may be incorporated into the controlled-release compositions of the invention. Suitable co-surfactants include, but are not limited to:
a) natural and synthetic lipophilic agents, e.g., phospholipids, cholesterol, and cholesterol fatty acid esters and derivatives thereof;
b) nonionic surfactants, which include for example, polyoxyethylene fatty alcohol esters, sorbitan fatty acid esters (Spans), polyoxyethylene sorbitan fatty acid esters (e.g., polyoxyethylene (20) sorbitan monooleate (Tween 80), polyoxyethylene (20) sorbitan monostearate (Tween 60), polyoxyethylene (20) sorbitan monolaurate (Tween 20) and other Tweens, sorbitan esters, glycerol esters, e.g., Myrj and glycerol triacetate (triacetin), polyethylene glycols, cetyl alcohol, cetostearyl alcohol, stearyl alcohol, polysorbate 80, poloxamers, poloxamines, polyoxyethylene castor oil derivatives (e.g., Cremophor® RH40, Cremphor A25, Cremphor A20, Cremophor® EL) and other Cremophors, sulfosuccinates, alkyl sulphates (SLS); PEG glyceryl fatty acid esters such as PEG-8 glyceryl caprylate/caprate (Labrasol), PEG-4 glyceryl caprylate/caprate (Labrafac Hydro WL 1219), PEG-32 glyceryl laurate (Gelucire 444/14), PEG-6 glyceryl mono oleate (Labrafil M 1944 CS), PEG-6 glyceryl linoleate (Labrafil M 2125 CS); propylene glycol mono- and di-fatty acid esters, such as propylene glycol laurate, propylene glycol caprylate/caprate; Brij® 700, ascorbyl-6-palmitate, stearylamine, sodium lauryl sulfate, polyoxethyleneglycerol triiricinoleate, and any combinations or mixtures thereof;
c) anionic surfactants include, but are not limited to, calcium carboxymethylcellulose, sodium carboxymethylcellulose, sodium sulfosuccinate, dioctyl, sodium alginate, alkyl polyoxyethylene sulfates, sodium lauryl sulfate, triethanolamine stearate, potassium laurate, bile salts, and any combinations or mixtures thereof;
d) cationic surfactants such as quarternary ammonium compounds, benzalkonium chloride, cetyltrimethylammonium bromide, and lauryldimethylbenzyl-ammonium chloride;
substituted cellulose derivatives such as methylcellulose, hydroxycellulose, hydroxyl propylcellulose, hydroxyl propylmethylcellulose, noncrystaslline cellulose, sodium carboxymethylcellulose and any combinations or mixtures thereof;
f) polyethylene glycol (PEG) 200 to 6000 and the like, Lutrol® F-127, Lutrol® F-88, Solutol® HS-15, poly (ethylene glycol) distearate, and any combinations or mixtures thereof.

Other surfactants suitable for use as a co-surfactant in the present invention include colloidal clays (e.g., bentonite and veegum), natural proteins (e.g., casein and gelatin), tragacanth, waxes, enteric resin, paraffins, acacia and any combinations and mixtures thereof.

In certain other preferred embodiments, the preferred co-surfactant is Lutrol F-127. Lutrol® F 127 is soluble in water, ethanol (95%) and isopropanol. It is insoluble in ether, paraffin and fatty oils. Lutrol F-127 is used primarily as a thickening agent and gel former, but also as a co-emulsifier and consistency enhancer in creams and liquid emulsions. It is also used as a solubilizer for certain active substances such as nifedipine, naproxen and fenticonazole as well as for essential oils in pharmaceutical and cosmetic formulations. Lutrol® F 127 is suitable for the formulation of active substances that show reduced solubility as a result of neutralization.

Concentrations of the oil-based surfactant alone or mixture of oil-based surfactant and additional co-surfactants contained in the controlled-release emulsion composition of the present invention may range from about 0.1% to about 99% w/v. In other embodiments, the concentrations may range from about 1% to about 50% and in other embodiments, from about 1% to about 20%.

When the solubilized material of the present invention contain a co-surfactant in addition to the oil-based surfactant, the ratio of oil-based surfactant to co-surfactant may range from about 1:1 to about 10:1.

In certain embodiments of the present invention, a mixture of Vitamin E-TPGS and Lutrol F-127 can be utilized. The ratio of Vitamin E-TPGS to Lutrol F-127 is from about 1:1 to about 10:1. In certain preferred embodiments, the ratio may be about 2:1. In yet another preferred embodiment, the ratio may be about 4:1.

Solubilizers

If the insoluble active agent is not soluble in the oil-based surfactant, than the emulsion should also contain a solubilizer in addition to the oil-based surfactant. The active agent may be dissolved in the solubilizer itself or together with the oil-based surfactant. Solvents suitable for dissolving the active agent include, but are not limited to, ethanol, propylene glycol, transcutol, glycerol, isopranpol, 2-pyrrolidone, N-methyl-2-pyrrolidone, polyethylene glycols, such as, but not limited to, PEG-200 to PEG 6000 and the like; mineral oil, safflower oil, olive oil, coconut oil, sesame oil, corn oil, castor oil, lemmon oil, peppermint oil, duoprime oil 70, soybean oil, lemon oil, peppermint oil, triacetin, glycofurol, propylene carbonate, dimethyl acetaminde, dimethyl isosorbide, and any combinations or mixtures thereof.

In certain embodiments, the preferred solvent is N-methyl-2-pyrrolidone (NMP). NMP is miscible in water at any ratio and has good stability, but has been known to decompose upon exposure to light.

In other embodiments, the preferred solvent is 2-pyrrolidone.

Other co-solvents suitable for use in the present invention include organic acids such as, but not limited to, succinic acid, ascorbic acid, oleinic acid, alginic acid, stearic acid, lenic acid, fumaric acid, and citric acid and the like.

The ratio of solvent to surfactant (oil-based surfactant or oil-based surfactant/co-surfactant) may range from about 1:1 to about 5:1. In certain preferred embodiments, the ratio may be about 2:1.

Controlled Release Carriers

The controlled-release carrier of the invention may comprise at least one pharmaceutically acceptable gelling agent. In other embodiments, the controlled-release carrier may comprise at least one pharmaceutically acceptable gelling agent and an inert diluent. In certain other embodiments, the controlled-release carrier may comprise two or more pharmaceutically acceptable gelling agents.

The pharmaceutically acceptable gelling agents of the present invention may be selected from the group consisting of heteropolysaccharide gum and a homopolysaccharide gum. Heterodisperse excipients, previously disclosed in our U.S. Pat. Nos. 4,994,276, 5,128,143, and 5,135,757, may be utilized in the controlled-release carrier of the present invention. For example, the controlled-release carrier comprises a gelling agent of both hetero- and homo-polysaccharides which exhibit synergism, e.g., the combination of two or more polysaccharide gums producing a higher viscosity and faster hydration than that which would be expected by either of the gums alone, the resultant gel being faster-forming and more rigid.

The term “heteropolysaccharide” as used in the present invention is defined as a water-soluble polysaccharide containing two or more kinds of sugar units, the heteropolysaccharide having a branched or helical configuration, and having excellent water-wicking properties and immense thickening properties. In certain embodiments, when admixed with an appropriate homopolysaccharide capable of enhancing the gel strength of the hydrophilic matrix upon exposure to an environmental fluid, e.g. gastric fluid, etc., the gum packs closely and many intermolecular attachments are formed which make the structure strong and provide a hydrophilic gum matrix having high gel strength. The homoploysaccharide is therefore an agent capable of enhancing the gel strength of a gel formed by exposure of the heteropolysaccharide to an environmental fluid, thus affecting the rate of release of the active agent from the composition.

One particular heteropolysacharide suitable for use in the present invention is xanthan gum.

Xanthan gum, the preferred heteropolysaccharide, is produced by microorganisms, for instance, by fermentation with the organism xanthomonas compestris. Most preferred is xanthan gum which is a high molecular weight (>10⁶) heteropolysaccharide. Xanthan gum contains D-glucose, D-mannose, D-glucuronate in the molar ratio of 2.8:2.0:20, and is partially acetylated with about 4.7% acetyl. Xanthan gum also includes about 3% pyruvate, which is attached to a single unit D-glucopyromosyl side chain as a metal. It dissolves in hot or cold water and the viscosity of aqueous solutions of xanthan gum is only slightly affected by changes in the pH of a solution between 1 and 11.

The homopolysaccharide gums used in the present invention include the galactomannans, i.e., polysaccharides which are composed solely of mannose and galactose. Galactomannans which have higher proportions of unsubstituted mannose are preferred in certain embodiments. Locust bean gum, which has a higher ratio of mannose to the galactose, is especially preferred as compared to other galactomannans such as guar and hydroxypropyl guar.

Other acceptable controlled-release carriers which may be used in the present invention include those gelling agents well-known in the art. Examples include vegetable gums such as alginates, gum karaya, pectin, agar, tragacanth, accacia, carrageenan, tragacanth, chitosan, agar, alginic acid, other polysaccharide gums (e.g. hydrocolloids), and mixtures of any of the foregoing. Further examples of specific gums which may be useful in the present invention include but are not limited to acacia catechu, salai guggal, indian bodellum, copaiba gum, asafetida, cambi gum, enterolobium cyclocarpum, mastic gum, benzoin gum, sandarac, gambier gum, butea frondosa (Flame of Forest Gum), myrrh, konjak mannan, guar gum, welan gum, gellan gum, tara gum, locust bean gum, carageenan gum, glucomannan, galactan gum, sodium alginate, tragacanth, chitosan, xanthan gum, deacetylated xanthan gum, pectin, sodium polypectate, gluten, karaya gum, tamarind gum, ghatti gum, Accaroid/Yacca/Red gum, dammar gum, juniper gum, ester gum, ipil-ipil seed gum, gum talha (acacia seyal), and cultured plant cell gums including those of the plants of the genera: acacia, actinidia, aptenia, carbobrotus, chickorium, cucumis, glycine, hibiscus, hordeum, letuca, lycopersicon, malus, medicago, mesembryanthemum, oryza, panicum, phalaris, phleum, poliathus, polycarbophil, propylene glycol alginate, sida, solanum, trifolium, trigonella, Afzelia africana seed gum, Treculia africana gum, detarium gum, cassia gum, carob gum, Prosopis africana gum, Colocassia esulenta gum, Hakea gibbosa gum, khaya gum, scleroglucan, zea, modified starch, hydroxypropylmethylcellulose, hydroxyethylcellulose, methylcellulose, and other cellulosic materials such as sodium carboxymethylcellulose and hydroxypropyl cellulose; acrylics, such as carbomer (cross-linked polyacrylic acid), acrylic acid copolymers; and mixtures of any of the foregoing, and the like. This list is not meant to be exclusive.

In certain embodiments, the heteropolysaccharide gum and the homopolysaccharide gum are in a weight ratio from about 1:20 to about a 20:1.

In preferred embodiments, the heteropolysaccharide gum comprises from about 1% to about 99% by weight of the controlled-release carrier. In other preferred embodiments, the homopolysaccharide gum comprises from about 1% to about 99% by weight of the controlled-release carrier. In another preferred embodiment, the total combined weight of the heteropolysaccharide and the homopolysaccharide is from about 65% to about 99% of the controlled-release carrier. In certain embodiments from about 20% to about 35% by weight of an inert diluent, e.g., microcrystalline cellulose is present in the controlled-release carrier.

Inert diluents suitable for incorporation into the controlled-release carrier may include any pharmaceutically acceptable inert diluent such as mannitol, sucrose, dextrose, lactose, microcrystalline cellulose, xylitol, fructose, sorbitol, mixtures and combinations thereof. Preferably, the inert diluent may comprise a monosaccharide, a disaccharide, a polyhydric alcohol, a cellulose (such as microcrystalline cellulose), starches, and any combinations or mixtures thereof. In certain embodiments, the inert diluent may be silicified microcrystalline cellulose.

In other preferred embodiments of the invention, the inert diluent may not be incorporated in to the controlled-release carrier, yet still be incoproated into the controlled-release composition. For instance, in certain embodiments, the inert diluent may be incorporated together with the solubilized material to obtain a wet granulation agent prior to granulation with the controlled-release carrier. In other embodiments, the inert diluent may be incorporated into the granulation after the solubilized material has been mixed together with the controlled-release carrier.

In certain other embodiments of the present invention, the controlled-release carrier may contain an ionizable gel strength enhancing agent. The ionizable gel strength enhancing agent may be monovalent, divalent or multivalent ionizable salts and any combinations or mixtures thereof. Preferred salts are the inorganic salts, including various alkali metal and/or alkaline earth metal sulfates, chlorides, borates, bromides, etc. Ionizable alkaline earth organic salts such as citrates, acetates, lactates, etc. may also be used in accordance with the present invention. Specific examples of suitable ionizable gel strength enhancing agents include calcium sulfate, sodium chloride, potassium sulfate, sodium carbonate, lithium chloride, tripotassium phosphate, sodium borate, potassium bromide, potassium fluoride, sodium bicarbonate, calcium chloride, magnesium chloride, sodium citrate, sodium acetate, calcium lactate, magnesium sulfate, alkali metal chlorides, magnesium chloride, calcium chloride, organic acids such as citric, succiniic, fumaric, malic, maleic, glutaric, lactic and the like, alkali metal sulfates such as sodium sulfate, alkali metal alkyl sulfates wherein the alkyl group is from 1 to 14 carbon atoms, such as sodium methyl sulfate, sodium lauryl sulfate and the like as well as dioctyl sodium sulfosuccinate, dihydrogen sodium phosphate, monohydrogen sodium phosphate, disodium hydrogen phosphate, sodium chloride, sodium fluoride and mixtures thereof. Multivalent metal cations may also be utilized. However, the preferred ionizable gel strength enhancing agents are bivalent. Particularly preferred salts are calcium sulfate and sodium chloride.

It is to be understood that the ionizable compound may be a single compound or a mixture of two or more materials that provide the desired release characteristics. The ionizable gel strength enhancing agents of the present invention are added in an amount effective to obtain a desirable increased gel strength of a gel formed by exposure of the heteropolysaccharide to an aqueous environment.

In other embodiments of the invention, the ionizable gel strength enhancing agent may comprise a surfactant or wetting agent such as pharmaceutically acceptable anionic surfactants, cationic surfactants, amphoteric (amphipathic/amphophilic) surfactants, and non-ionic surfactants. Examples of suitable surfactant or wetting agents include alkali metal chlorides, magnesium chloride, calcium chloride, organic acids such as citric, succinic, fumaric, malic, maleic, glutaric, lactic and the like, alkali metal sulfates such as sodium sulfate, alkali metal alkyl sulfates wherein the alkyl group is from 1 to 14 carbon atoms, such as sodium methyl sulfate, sodium lauryl sulfate and the like as well as dioctyl sodium sulfosuccinate, dihydrogen sodium phosphate, monohydrogen sodium phosphate, disodium hydrogen phosphate, sodium chloride, sodium fluoride and mixtures thereof. It is to be understood that the ionizable compound may be a single compound or a mixture of two or more materials that provide the desired release characteristics. Other examples of suitable surfactants and/or suitable wetting agents are disclosed in U.S. Pat. No. 5,478,574, assigned to the assignee of the present invention. When the controlled-release carrier contains a surfactant as an ionizable gel strength enhancing agent, the surfactant can be different from or the same as the co-surfactant utilized in the emulsion.

The ionizable gel strength enhancing agent may comprise from about 1 to about 20% by weight of the controlled-release excicpient.

The skilled artisan will understand that the above ratios will vary according to, e.g., the type of heteropolysacccharide, homopolysaccharide and ionizable gel strength enhancing agent or combinations thereof are present in the composition, so long as the desired dissolution parameters of the composition is maintained.

In certain other embodiments, the ionizable gel-strength enhancing agent may be incorporated into the controlled-release composition without being contained in the controlled-release carrier. For example, in certain embodiments, the ionizable gel-strength enhancing agent may be incorporated into the composition after the controlled-release carrier and emulsion have been mixed.

In certain other embodiments of the invention, the controlled-release carrier may also include a hydrophobic material in an amount effective to slow the hydration of the gum without disrupting the hydrophilic matrix formed by the heteropolysaccharide when the formulation is exposed to fluids in an environment of use. This may be accomplished by granulating the controlled-release carrier with a solution or dispersion of hydrophobic material prior to the incorporation of the emulsion. The hydrophobic material may be selected from alkylcelluloses, acrylic and/or methacrylic acid polymers or copolymers, hydrogenated vegetable oils, zein, insoluble salts as well as other pharmaceutically acceptable hydrophobic materials known to those skilled in the art. A preferred hydrophobic cellulosic material is ethylcellulose. The amount of hydrophobic material incorporated into the controlled-release carrier may be that which is effective to slow the hydration of the gums without disrupting the hydrophilic matrix formed upon exposure to an environmental fluid, e.g. a gastric fluid.

When the hydrophobic material chosen is a pharmaceutically acceptable acrylic polymer, the acrylic polymer can include, but is not limited to, acrylic acid and methacrylic acid copolymers, methyl methacrylate copolymers, ethoxyethyl methacrylates, cynaoethyl methacrylate, poly(acrylic acid), poly(methacrylic acid), methacrylic acid alkylamide copolymer, poly(methyl methacrylate), polymethacrylate, poly(methyl methacrylate) copolymer, polyacrylamide, aminoalkyl methacrylate copolymer, poly(methacrylic acid anhydride), glycidyl methacrylate copolymers and any combinations or mixtures thereof.

In certain preferred embodiments, the acrylic polymer may be comprised of one or more ammonio methacrylate copolymers. Ammonio methacrylate copolymers are well known in the art, and are described in NF XVII as fully polymerized copolymers of acrylic and methacrylic acid esters with a low content of quaternary ammonium groups.

In order to obtain a desirable dissolution profile, it may be necessary to incorporate two or more ammonio methacrylate copolymers having differing physical properties, such as different molar ratios of the quaternary ammonium groups to the neutral (methacrylic) esters.

Certain methacrylic acid ester-type polymers are useful for preparing pH-dependent coatings which may be used in accordance with the present invention. For example, there are a family of copolymers synthesized from diethylaminoethyl methacrylate and other neutral methacrylic esters, also known as methacrylic acid copolymer or polymeric methacrylates, commercially available as Eudragit® from Degussa, Inc. There are several different types of Eudragit®. For example, Eudragit E is an example of a methacrylic acid copolymer which swells and dissolves in acidic media. Eudragit L is a methacrylic acid copolymer which does not swell at about pH<5.7 and is soluble at about pH>6. Eudragit S does not swell at about pH<6.5 and is soluble at about pH>7. Eudragit RL and Eudragit RS are water swellable, and the amount of water absorbed by these polymers is pH-dependent, however, compositions coated with Eudragit RL and RS are pH-independent.

In certain embodiments of the present invention, the hydrophobic material may be included in the controlled-release carrier in an amount from about 1% to about 20% by weight. More preferably, the hydrophobic material may be included in the controlled-release carrier in an amount from about 3% to about 12%, and most preferably from about 5% to about 10%, by weight of the final composition. The hydrophobic material may be dissolved in an organic solvent or dispersed in an aqueous solution for incorporation into the formulation.

The controlled-release carrier of the present invention preferably have uniform packing characteristics over a range of different particle size distributions and are capable of processing into the final composition (e.g., tablets) using either direct compression, following addition of drug and lubricant powder, or conventional wet granulation.

In certain embodiments, the properties and characteristics of a specific carrier system prepared according to the present invention are dependent in part on the individual characteristics of the homo and heteropolysaccharide constituents, in terms of polymer solubility, glass transition temperatures etc., as well as on the synergism both between different homo- and heteropolysaccharides and between the homo and heteropolysaccharides and the inert saccharide constituent(s) in modifying dissolution fluid-carrier interactions.

The combination of the gelling agent (e.g., a mixture of xanthan gum and locust bean gum) with the inert diluent, with or without the ionizable gel strength enhancing agent and hydrophobic polymer, provides a ready-to-use controlled-release carrier product in which a formulator need only blend the desired active medicament, an optional wetting agent, an optional pH modifying agent, an optional surfactant and an optional lubricant with the carrier before compressing the mixture to form slow release tablets. The carrier may comprise a physical admix of the gums along with a soluble carrier such as compressible sucrose, lactose or dextrose, although it is preferred to granulate or agglomerate the gums with plain (i.e., crystalline) sucrose, lactose, dextrose, etc., to form a carrier. The granulate form has certain advantages including the fact that it can be optimized for flow and compressibility; it can be tableted, formulated in a capsule, extruded and spheronized with an active medicament to form pellets, etc.

In general, the formulation may be prepared as a directly compressible diluent, for example, by wet granulating, spray drying lactose or as a premixed direct compression diluent by art known methods. For purposes of the present invention, these specially treated inert diluents will be referred to as “directly compressible” inert diluents or excipients.

Direct Compression Excipients

In certain other embodiments, the controlled-release compositions of the present invention may further comprise a pre-manufactured direct-compression excipient. Examples of such pre-manufactured direct compression excipients include Prosolv® (silicified microcrystalline cellulose), Emcocel® (microcrystalline cellulose, N.F.), Emdex® (dextrates, N.F.), and Tab-Fine® (a number of direct-compression sugars including sucrose, fructose, and dextrose), all of which are commercially available from JRS Pharma Inc., Patterson, N.Y.). Other direct compression diluents include Anhydrous lactose (Lactose N.F., anhydrous direct tableting) from Sheffield Chemical, Union, N.J. 07083; Elcems® G-250 (Powdered cellulose, N.F.) from Degussa, D-600 Frankfurt (Main) Germany; Fast-Flo Lactose® Lactose, N.F., spray dried) from Foremost Whey Products, Banaboo, Wis. 53913; Maltrin® (Agglomerated maltrodextrin) from Grain Processing Corp., Muscatine, Iowa 52761; Neosorb 60® (Sorbitol, N.F., direct-compression) from Roquette Corp., 645 5th Ave., New York, N.Y. 10022; Nu-Tab® (Compressible sugar, N.F.) from Ingredient Technology, Inc., Pennsauken, N.J. 08110; Poly plasdone XL® (Crospovidone, N.F., cross-linked polyvinylpyrrolidone) from ISP Corp, Wayne N.J. 07470; Primojel® (Sodium starch glycolate, N.F., carboxymethyl starch) from Generichem Corp., Little Falls, N.J. 07424; Solka Floc® (Cellulose floc) from JRS Pharmaceuticals, Inc., Patterson, N.Y. 12563, Spray-dried lactose® (Lactose N.F., spray dried) from Foremost Whey Products, Baraboo, Wis. 53913 and DMV Corp., Vehgel, Holland; Cabosil® from Cabot Co.; and Sta-Rx 1500® (Starch 1500) (Pregelatinized starch, N.F., compressible) from Colorcon, Inc., West Point, Pa. 19486. Pre-manufactured directed compression excipients may also comprise all or a portion of the inert diluent.

In further embodiments of the present invention, the directly compressible inert diluent which is used in conjunction with the controlled-release pharmaceutical carrier of the present invention is an augmented microcrystalline cellulose as disclosed in U.S. Pat. No. 5,585,115, issued on Dec. 17, 1996, hereby incorporated by reference in its entirety. The augmented microcrystalline cellulose described therein is commercially available under the tradename “Prosolv” from JRS Pharma, Inc.

Additional Ingredients

In certain other embodiments of the present invention, a pH modifying agent may be included in the composition. When a pH modifying agent is included in the composition, preferably it is present from about 0.5% to about 10% by weight of the final composition and the pH modifying agent facilitates the release of the drug from the matrix. In certain embodiments, the pH modifying agent preferably facilitates the release of the active agent or pharmaceutically acceptable salt thereof by the formulation to provide high bioavailability. In certain embodiments, the pH modifying agent is an acid, preferably an organic acid such as citric acid, succinic acid, fumaric acid, malic acid, maleic acid, glutaric acid, lactic acid, and the like. In certain embodiments, the pH modifying is a base. Suitable inorganic bases include sodium hydroxide, potassium hydroxide and carbonates and bicarbonates of sodium and potassium and other suitable elements, and the like. Suitable organic bases include propanolamine, ethanolamine, methylamine, dimethyl formamide, dimethylacetamide, diethanolamine, diisopropanolamine, triethanolamine, and the like.

Other acceptable ingredients may optionally be included in controlled-release compositions of the present invention such as, but not limited to, vegetable gums, alginates, carrageenan, pectin, guar gum, and modified starch.

In other embodiments of the present invention, the controlled-release compositions of the present invention can contain complexants. Complexants may be utilized when the compositions of the present invention contain a low dose of insoluble active agent. Precipitation of an insoluble drug from a composition during its shelf life has been a problem. Complexation agents act to further solubilize water insoluble drugs, thus preventing any precipitation from the composition upon storage. Suitable complexants include, but are not limited to, cyclodextrins, polyethylene glycols, crosslinked polyvinyl pyrrolidone, polyvinyl polypyrrolidone, methacrylates and any combinations or mixtures thereof.

Dosage Forms

A. Oral Dosage Forms

The controlled-release oral compositions of the present invention can be manufactured as a suitable tablet or multiparticulate formulation utilizing procedures known to those skilled in the art which can be modified such that the composition provides for the release of the active agent or pharmaceutically acceptable salt thereof over about 12 to about 24 hours when exposed to an environmental fluid. In either case, the controlled-release composition includes a controlled-release carrier which is incorporated into a matrix along with the drug (e.g., carvedilol), or which is applied as a controlled release coating.

An oral composition according to the invention may be provided as, for example, granules, spheroids, beads, pellets (hereinafter collectively referred to as multiparticulates) and/or particles. An amount of the multiparticulates which is effective to provide the desired dose of active agent over time may be placed in a capsule or may be incorporated in any other suitable oral solid form. In one preferred embodiment of the present invention, the controlled release composition comprises such particles containing or comprising the active ingredient, wherein the particles have diameter from about 0.1 mm to about 2.5 mm.

Examples of suitable multiparticulate formulations are those in which the particles comprise inert beads which may be coated with the active agent emulsion. Thereafter, a coating comprising the controlled-release carrier is applied onto the beads. Alternatively, a spheronizing agent, together with the active agent emulsion can be spheronized to form spheroids. In such embodiments, in addition to drug and spheronizing agent, the spheroids may also contain a binder. Additionally (or alternatively) the spheroids may contain a water insoluble polymer, especially an acrylic polymer, an acrylic copolymer, such as a methacrylic acid-ethyl acrylate copolymer, or ethyl cellulose.

In certain embodiments, the particles comprise normal release matrixes containing the active agent emulsion. These particles are then coated with the controlled-release carrier (e.g., controlled-release coating).

In certain embodiments, coatings are provided to permit either pH-dependent or pH-independent release, e.g., when exposed to gastrointestinal fluid. Coatings which are pH-dependent may be used in accordance with the present invention include shellac, cellulose acetate phthalate (CAP), polyvinyl acetate phthalate (PVAP), hydroxypropylmethylcellulose phthalate, and methacrylic acid ester copolymers, and the like.

In other embodiments, the tablet core or multiparticulates may be coated with one or more of the hydrophobic materials discussed above. The coating may be applied in the form of an organic or aqueous solution or dispersion. The coating may be applied to obtain a weight gain from about 2% to about 25% of the substrate in order to obtain a desired controlled-release profile.

The compositions can be administered to a human or animal orally via encapsulation in a soft or hard gelatin capsule or by compression into a tablet.

When the composition is a tablet, a complete mixture of the emulsion and controlled-release carrier, in an amount sufficient to make a uniform batch of tablets, may be subjected to tableting in a conventional production scale tableting machine at normal compression pressure, i.e. about 2000-6000 lbs/sq/in. However, the mixture should not be compressed to such a degree that there is subsequent difficulty in its hydration when exposed to gastric fluid. An effective amount of any generally accepted pharmaceutical lubricant, including the calcium or magnesium soaps may be added to the above-mentioned ingredients prior to compression intotablets. One preferred lubricant is Pruv®, e.g., in the amount of about 3.0 percent of the solid composition.

When the controlled-release compositions of the present invention are tablets, the tablets may be optionally coated with an overcoat containing, e.g., hydroxypropylmethylcellulose, colorants and the like.

Variables which may affect the release rate of the compositions and the compressibility of tablets prepared with the controlled-release carrier of the present invention are the drug to gum ratio; the method of incorporation of controlled-release carrier (method of granulation); the relative amount of gelling agent, e.g., the heteropolysaccharide gum to homopolysaccharide gum and additional ingredients, such as hydrophobic material; the ratio of emulsion to the controlled-release carrier; and the types of surfactant and optional co-surfactants utilized.

In certain embodiments, the controlled-release compositions of the present invention may release from about 1% to about 10% of the active agent by weight at about 1 hour after exposure of the composition to an environmental fluid and provide controlled-release of the active agent for a period of about 12 to about 24 hours. Most preferably the compositions of the present invention may provide a release of about 1% to about 10% after 1 hour, from about 4% to about 12% after 3 hours, from about 8% to about 20% after 6 hours, from about 20% to about 70% after 10 hours, from about 50% to about 95% after 16 hours and from about 70% to about 100% after 24 hours.

B. Transdermal Delivery Systems

The dosage forms of the present invention may also be manufactured as suitable transdermal delivery systems utilizing procedures known to those skilled in the art which can be modified such that the transdermal delivery system provides for the release of the active agent or pharmaceutically acceptable salt thereof over about 12 hours to about 7 days after application to a patient in need thereof. In either case, the transdermal composition includes a solubilized material comprising an active agent and at least one oil-based surfactant capable of solubilizing the active agent, and an optional controlled release material. When the transdermal delivery system contains a controlled release material, the solubilized material may be dispersed in the controlled release material.

The transdermal delivery systems of the present invention may also contain any/all of the additional ingredients described above with regard to the oral compositions, such as, but not limited to solubilizers, surfactants, co-surfactants, pH modifiers, and the like.

Suitable transdermal delivery systems for use in the present invention include, but are not limited to, transdermal patches, transmucousal delivery systems, creams, ointments, and gels, pastes, lotions and the like. (See: Introduction to Pharmaceutical Dosage Forms, Ansel, Howard C., 4^{th} Edit., Chap. 11, pp. 291-320 (1985) and Remington, The Science and Practice of Pharmacy, Gennaro, Alfonso R. et al., 20^{th} Edit., Chap. 47, pp. 917-925, (2000) the disclosures of which are hereby incorporated by reference.)

In certain embodiments of the present invention, the transdermal delivery system is a transdermal patch comprising an active agent dispersed in an emulsion, the emulsion comprising at least one oil-based surfactant and an optional solubilizer and/or co-surfactant contained in a reservoir or a matrix, and an adhesive which allows the transdermal delivery system to adhere to the skin, allowing the passage of the active agent from the transdermal system through the skin of the patient. Once the active agent has penetrated the skin layer, the active is absorbed into the blood stream where it can exert desired pharmaceutical effects. The transdermal patches of the present invention release the active agent in a controlled-release manner, such that the blood levels of the active agent is maintained at therapeutically effective level through out the dosing period.

A transdermal patch can consist several layers: in the inner side a peelable plastic cover will protect the drug layer containing the adhesive polymer, plasticizer, the oxidizing agents, penetration enhancers and other excipients. The outer layers (i.e., the external layers) are designated to protect the drug from diffusion outward and to stick the patch by its margins to the skin, so the drug layer is occluded from all sides except the skin side where it is in close contact (see, e.g., Chien, Y. W., Transdermal Controlled Systemic Medications, 1987, Marcel & Decker, pp. 93-120, 365-378).

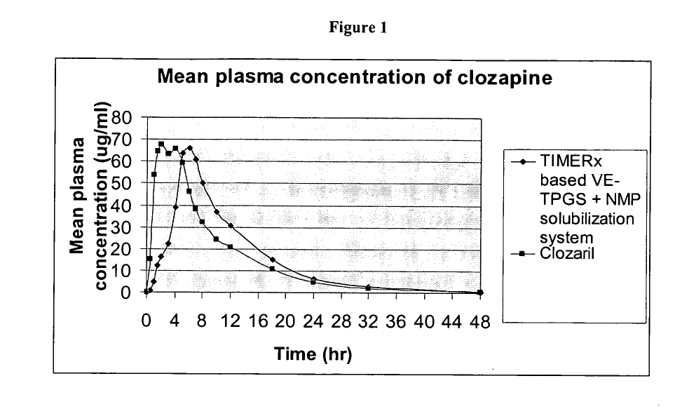

There are two basic types of transdermal patches that may be used to deliver the active agent emulsion described above. One is a liquid reservoir patch in which the active agent emulsion, is confined in a pouch or sac within the device. An example of such a device for delivering is shown in FIG. 1 of U.S. Pat. No. 5,364,630. The other is a matrix patch in which the active agent emulsion is dissolved in one or more polymeric layers of a laminated composite. An example of this type of matrix patch is described in U.S. Pat. No. 5,603,947.

In the manufacture of matrix patches for administering the active agent emulsion, it is common to attempt to avoid steps involving heat treatment, e.g., drying, so as to avoid excessive loss or degradation of the active agent. For instance U.S. Pat. Nos. 4,915,950 and 5,603,947, the disclosures of which are hereby incorporated by reference, describe a printing procedure whereby the active agent, neat nicotine, is applied to a nonwoven fabric laminated to a polyisobutylene adhesive layer. Alternatively “hot” melt adhesives that melt at relatively low temperatures have been used as a matrix material for these drugs. See U.S. Pat. No. 5,411,739, the disclosure of which is hereby incorporated by reference.

Methods of Preparation

A. Emulsion Preparation

The emulsion of the present invention may be prepared utilizing different solubilization techniques as well as various granulation processes. For example, a solute modification technique may be utilized that results in modification of the crystalline structure and an increase in surface area; creation of a more soluble (and less stable) polymorph. In certain embodiments, the solute modification technique contemplates the addition of a cosolvent. Cosolvents useful in the present invention include, but are not limited to, alcohols, propylene glycol, polyethylene glycols (e.g., PEG-400), 2-pyrrolidone (Soluphor-P), N-methyl-2-pyrrolidone, or NMP (Pharmasolve®), glycerin, dimethyl sorbide and any combinations or mixtures thereof. Here, the insoluble active agent is dissolved in a solvent. The active agent/solvent solution is then admixed with an oil-base surfactant to provide a emulsion. The emulsion is then mixed together with a controlled-release carrier, which mixture can then be incorporated into a capsule or compressed into tablets to provide for the controlled-release compositions of the present invention.

In certain embodiments, the emulsion may be prepared by completely dissolving the desired amount of active agent in solvent, e.g., N-methyl-2-pyrrolidine. Next, an amount of oil-based surfactant, e.g., Vitamin E TPGS can be added to the active agent solution. Once the emulsion is formed, the particle size can measured to determine that the particles are in a preferred droplet size between 7.8 and 20 nm.

In certain other embodiments, the solvent and oil-based surfactant can be combined together to form a surfactant solubilization system, prior to the addition of the active agent.

The emulsion may be further diluted into a pharmaceutically acceptable vehicle prior to being combined with the controlled-release carrier. A water-in-oil emulsion can be further diluted or extended with an oleaginous or oil-miscible liquid, whereas an oil-in-water emulsion can be further diluted or extended with water or some other aqueous medium.

In certain embodiments, an inert diluent may be incorporated into the controlled-release composition together with the emulsion prior to granulating the emulsion with the controlled-release carrier. For example, in certain embodiments, a wet granulation of the inert diluent and emulsion may be prepared prior to mixing with the controlled-release ecipient.

B. Preparation of the Controlled-Release Carrier

The controlled-release carrier of the present invention may comprise at least one pharmaceutically acceptable gelling agent. In other embodiments, the controlled-release carrier can be prepared by admixing an effective amount of a gelling agent, e.g., a heteropolysaccharide and/or homopolysaccharide together with an inert diluent and, optionally, an ionizable gel strength enhancing agent.

The combination of the gelling agent and inert diluent provides a ready to use controlled-release carrier in which a formulator need only blend the desired amount of emulsion to form a granulate and then incorporate the granulate into an oral solid composition.

In certain embodiments, the controlled-release carrier may thus comprise a physical admix of the heteropolysaccharide with the homoploysaccharide and inert diluent.

The controlled-release carrier of the present invention may be prepared in accordance with any granulation technique to yield an acceptable carrier product. In certain embodiments, the controlled-release carrier of the present invention may be prepared via wet granulation techniques prior to mixing with the emulsion. In this technique, the desired amounts of gelling agent(s) and optional inert diluent (and other excipients) may be mixed together and moistened with a wet granulating aid such as water, propylene glycol, glycerol, alcohol or the like to prepare a moistened mass. The moistened mass may be dried, and then milled with, e.g., conventional equipment, into granules. The resultant controlled-release carrier is ready to use. The controlled-release carrier may have certain advantages including it is free-flowing, good cohesive properties, and can be directly admixed with the emulsion, e.g., via wet granulation, and formed into the desired composition such as a tablet. On the other hand, the mixture of controlled-release carrier and emulsion may be formulated into a capsule.

In certain preferred embodiment where the controlled-release carrier is pre-manufactured , the controlled-release carrier is preferably free-flowing and directly compressible.

C. Preparation of a Controlled-Release Tablet

Once the emulsion has been prepared and the preferred droplet size is obtained, the next step in the preparation of the controlled-release compositions of the present invention is to combine the emulsion with the controlled-release carrier and other ingredients.

In certain embodiments, the emulsion and the controlled-release carrier may be mixed together via standard granulation techniques known in the art. In certain embodiments the emulsion and the controlled-release carrier may be mixed together via wet granulation utilizing a wet granulating aid, e.g. water or ethyl alcohol. The resultant mixture may then be dried, e.g. in a fluid bed dryer, and compressed into tablets.

In certain other embodiments, the emulsion may be added as a wet granulation agent to the controlled-release carrier to form wet granules. The granules may then dried utilizing standard drying techniques and passed through a sieve. The resulting granules may then blended with additional active agent and a lubricant and the lubricated granules compressed into tablets.

In certain other embodiments, a direct compression excipient may be premixed together with the controlled-release carrier prior to the addition of the emulsion.

In other embodiments, the emulsion and controlled-release carrier may be mixed together via dry granulation techniques and compressed together into tablets. In alternative preferred embodiments, the individual ingredients of the controlled-release carrier may be wet granulated with the emulsion.

When the final product to be manufactured is tablets, the complete mixture, in an amount sufficient to make a uniform batch of tablets, is then subjected to tableting in a conventional production scale tableting machine at normal compression pressure, i.e. about 2000-1600 lbs/sq in. However, the mixture should not be compressed to such a degree that there is subsequent difficulty in its hydration when exposed to gastric fluid.

One of the limitations of direct compression as a method of tablet manufacture is the size of the tablet. If the amount of active is high a pharmaceutical formulator may choose to wet granulate the active with other excipients to attain a decent size tablet with the right compact strength. Usually the amount of filler/binder or excipients needed in wet granulation is less than that in direct compression since the process of wet granulation contributes to some extent toward the desired physical properties of a tablet.

In certain embodiments, the average particle size of the granulated excipient of the present invention ranges from about 50 microns to about 400 microns and preferably from about 185 microns to about 265 microns. The particle size of the granulation is not narrowly critical, the important parameter being that the average particle size of the granules, must permit the formation of a directly compressible excipient which forms pharmaceutically acceptable tablets. In certain embodiments, the desired tap and bulk densities of the granulation of the present invention are normally between from about 0.3 to about 0.8 g/ml, with an average density of from about 0.5 to about 0.7 g/ml. Preferably, the tablets formed from the granulations of the present invention are from about 5 to about 20 kg hardness. In certain embodiments, the average flow of the granulations prepared in accordance with the present invention are from about 25 to about 40 g/sec. Tablets compacted using an instrumented rotary tablet machine have been found to possess strength profiles which are largely independent of the inert saccharide component. Scanning electron photomicrographs of largely tablet surfaces have provided qualitative evidence of extensive plastic deformation on compaction, both at the tablet surface and across the fracture surface, and also show evidence of surface pores through which initial solvent ingress and solution egress may occur.

An effective amount of a wetting agent may also be included in the formulation in order to increase the bioavailability of the active agent.

Suitable wetting agents for use in conjunction with the present invention include polyethyleneglycols as esters or ethers. Examples include polyethoxylated castor oil, polyethoxylated hydrogenated castor oil, polyethoxylated fatty acid from castor oil or polyethoxylated fatty acid from castor oil or polyethoxylated fatty acid from hydrogenated castor oil. Commercially available wetting agents which can be used are known under trade names Cremophor, Myrj, Polyoxyl 40 stearate, Emerest 2675, Lipal 395 and PEG 3350. An especially preferred wetting agent is polyethyleneglycol having a molecular weight of 3,350 (i.e., PEG 3350).

When included in the formulation, the wetting agent may be dissolved in a suitable solvent such as water, and thereafter added to the blended mixture of the controlled-release carrier and the emulsion. This allows the wetting agent to wet the particles of the carrier such that when the active is released from the emulsion the active drug particles released are tiny and do not aggregate.

The wetting agent may preferably be included in an amount effective to provide a final controlled-release product having acceptable bioavailability. For example, in certain embodiments of the present invention, the wetting agent may be included in an amount from about 5% to about 10% of the final product, by weight.

In further embodiments, the composition may be coated with a film coating e.g., a hydrophilic coating, in addition to or instead of the above-mentioned coatings. An example of a suitable material which may be used is hydroxypropylmethylcellulose (e.g., Opadry® as described above). The film coating of the present invention should be capable of producing a strong, continuous film that is smooth and elegant, capable of supporting pigments and other coating additives, non-toxic, inert, and tack-free.

Additionally, the compressed tablets may optionally be coated with a color coat that rapidly disintegrates or dissolves in water or the environment of use. The color coat may be a conventional sugar or polymeric film coating which is applied in a coating pan or by conventional spraying techniques. Preferred materials for the color coat are commercially available under the Opadry tradename (e.g, Opadry II® White). The color coat may be applied directly onto the tablet core, or may be applied after a coating as described above. Generally, the color coat surrounding the core will comprise from about 1 to about 5% preferably about 2 to about 4% based on the total weight of the tablet.

An effective amount of any generally accepted pharmaceutical lubricant or mixture of lubricants, including the calcium or magnesium soaps may be added to the above-mentioned ingredients of the formulation at the time the medicament is added, or in any event prior to compression into a solid composition. An example of a suitable lubricant is magnesium stearate in an amount of about 0.3% to about 3% by weight of the solid composition. An especially preferred lubricant is sodium stearyl fumarate, NF, commercially available under the trade name Pruv®. Other preferred lubricants include magnesium stearate and talc.

An effective amount of any generally acceptable pharmaceutical glidant or mixture of glidants may also be added to the above-mentioned ingredients of the formulation at the time the medicament is added, or in any event prior to compression into a solid composition. Glidants for use in the present invention include, for example, colloidal silicon dioxide, talc, silicon dioxide, sodium aluminosilicate, calcium silicate, powdered cellulose, microcrystalline cellulose, corn starch, sodium benzoate, calcium carbonate, magnesium carbonate, metallic stearates, calcium stearate, magnesium stearate, zinc stearate, stearowet C, starch, starch 1500, magnesium lauryl sulfate, magnesium oxide, and mixtures thereof.

In certain embodiments, additional inert diluent may also be incorporated in the controlled-release oral composition when mixing the controlled-release carrier with the torsemide or pharmaceutically acceptable salt thereof. The inert diluent may be the same or different inert diluent that is incorporated into the controlled-release carrier. Other pharmaceutically acceptable diluents and excipients that may be used to formulate oral compositions of the present invention are described in the Handbook of Pharmaceutical Excipients, American Pharmaceutical Association (1986).

In additional embodiments of the present invention, a support platform is applied to the tablets manufactured in accordance with the present invention. Suitable support platforms are well known to those skilled in the art. An example of suitable support platforms is set forth, e.g., in U.S. Pat. No. 4,839,177, hereby incorporated by reference. In that patent, the support platform partially coats the tablet, and consists of a polymeric material insoluble in aqueous liquids. The support platform may, for example, be designed to maintain its impermeability characteristics during the transfer of the therapeutically active medicament. The support platform may be applied to the tablets, e.g., via compression coating onto part of the tablet surface, by spray coating the polymeric materials comprising the support platform onto all or part of the tablet surface, or by immersing the tablets in a solution of the polymeric materials.

The support platform may have a thickness of, e.g., about 2 mm if applied by compression, and about 10 μm if applied via spray-coating or immersion-coating. Generally, in embodiments of the invention wherein a hydrophobic polymer or enteric coating is applied to the tablets, the tablets are coated to a weight gain from about 1 to about 20%, and in certain embodiments preferably from about 5% to about 10%.

Materials useful in the hydrophobic coatings and support platforms of the present invention include derivatives of acrylic acid (such as esters of acrylic acid, methacrylic acid, and copolymers thereof) celluloses and derivatives thereof (such as ethylcellulose), polyvinylalcohols, and the like.

In certain embodiments of the present invention, the tablet core includes an additional dose of the medicament included in either the hydrophobic or enteric coating, or in an additional overcoating coated on the outer surface of the tablet core (without the hydrophobic or enteric coating) or as a second coating layer coated on the surface of the base coating comprising the hydrophobic or enteric coating material.

The coatings of the present invention may be applied in any pharmaceutically acceptable manner known to those skilled in the art. For example, in one embodiment, the coating is applied via a fluidized bed or in a coating pan. The solvent for the hydrophobic polymer or enteric coating may be organic, aqueous, or a mixture of an organic and an aqueous solvent. The organic solvents may be, e.g., isopropyl alcohol, ethanol, methylene chloride, acetone, and the like, with or without water.

In certain preferred embodiments of the present invention, the controlled-release composition includes an immediate release component which comprises an effective amount of active agent or pharmaceutically acceptable salt thereof. In such embodiments, an effective amount of the active agent in immediate release form may be coated onto the multiparticulates or tablets of the present invention. For example, where the extended release of active agent from the formulation is due to a controlled release coating, the immediate release layer would be overcoated on top of the controlled release coating. On the other hand, the immediate release layer may be coated onto the surface of multiparticulates or tablets wherein the active agent is incorporated in a controlled release matrix. Where a plurality of the controlled-release multiparticulates comprising an effective unit dose of the torsemide or pharmaceutically acceptable salt thereof are incorporated into a capsule, the immediate release portion of the active agent dose may be incorporated into the capsule via inclusion of a sufficient amount of immediate release the active agent as a powder or granulate within the capsule. Alternatively, the capsule itself may be coated with an immediate release layer of the active agent.

In preferred embodiments, wherein the oral composition includes the active agent or pharmaceutically acceptable salt thereof in immediate release component, the oral composition is in the form of a bilayer tablet including a controlled-release portion and an immediate release portion. Preferably the immediate release portion comprises the active agent or a pharmaceutically acceptable salt thereof in combination with an immediate release excipient which may include any of the ingredients described herein with respect to the controlled-release oral composition, however, the ingredients are in an amount which allows for the immediate release of the active agent or pharmaceutically acceptable salt thereof upon exposure to an environmental fluid. For example, in certain embodiments, the immediate release portion of the bilayer oral composition may optionally include a gelling agent as described herein, a pharmaceutically acceptable diluent such as microcrystalline cellulose, and other pharmaceutically acceptable excipients described above (e.g., lubricant, diluent, wetting agent, pH modifying agent, surfactants, and the like), in an amount such that the active agent is able to release in an immediate release manner from the composition.

In certain preferred embodiments, the present invention is further directed to a method for preparing a controlled-release bilayer composition, comprising preparing a first layer comprising a controlled-release carrier comprising a gelling agent, ionizable gel strength enhancing agent, and pharmaceutically acceptable inert diluent. Thereafter a granulation solution optionally comprising a wetting agent and pH-modifying agent is added to the first portion of controlled-release carrier and granulated. The granulation is then dried and milled. An optional glidant is added to the blend. Thereafter, an opitional lubricant is added. The second layer of the bilayer composition is prepared by combining an immediate release excipient optionally comprising a gelling agent, optionally an ionizable gel strength enhancing agent, and a pharmaceutically acceptable inert diluent with an effective amount of active agent. Thereafter, an optional glidant is added and blended. An optional lubricant is then added and blended. The two layers are dispensed into separate hoppers of a bilayer tablet press and compressed.

The inclusion of an immediate release form of active agent or pharmaceutically acceptable salt thereof may be desired when, for example, a loading dose of a therapeutically active agent is needed to provide therapeutically effective blood levels of the active agent when the formulation is first exposed to gastric fluid. The loading dose of medicament included in the coating layer, the immediate release layer of the bilayer composition may be, e.g., from about 10% to about 40% of the total amount of medicament included in the formulation.

One skilled in the art would recognize still other alternative manners of incorporating the active agent in the immediate release or controlled-release portion of the unit dose. Such alternatives are deemed to be encompassed by the appended claims.

In certain embodiments, a second therapeutically effective agent may be included in the controlled-release oral compositions of the present invention. Preferably, the second therapeutic agent is also useful for the treatment of disorders disclosed herein. Such secondary drugs include for example and without limitation anti-hypertensive agents (e.g., ACE-inhibitors, calcium channel blockers, alpha-adrenergic blockers, beta-adrenergic blockers, and the like), other diuretics (e.g., loop-diuretics, thiazide diuretics, potassium sparing diuretics), digitalis glucosides, organic nitrates, combinations thereof, and the like. The second agent may be included in controlled release form or in immediate release form. In certain embodiments, the secondary drug is incorporated into the controlled release matrix along with the torsemide or a pharmaceutically acceptable salt thereof, is incorporated as a powder, granulation, etc. in the composition, or is incorporated into the controlled release oral composition in a coating on the composition.

### DETAILED DESCRIPTION OF THE PREFERRED EMBODIMENTS

The following examples illustrate various aspects of the present invention. They are not meant to be construed to limit the claims in any manner whatsoever. Preparation of TIMERx® Controlled-Release Carrier

The controlled release carrier utilized in this embodiment of the present invention is Applicants own TIMERx® technology. The controlled-release carrier is prepared as set forth below:

### EXAMPLE 1

### TIMERx® Excipients A, B, AND C

| Ingredients (%) | Excipient A | Excipient B | Excipient C |
|---|---|---|---|
| Xanthan gum | 35 | 5 | 65 |
| Locust bean gum | 35 | 65 | 5 |
| Dextrose | 30 | 30 | 30 |
| Water* | 28% | 37% | 21% |

*Water is removed during processing

The TIMERx® excipients A-C are prepared by the following steps:
1. Weigh out xanthan gum, locust bean gum and dextrose.
2. Charge high shear mixer/granulator with xanthan gum, locust bean gum and dextrose and dry blend for 3 minutes.
3. Add water and granulate until desirable granules are formed.
4. Dry granules in fluid bed dryer at 70° C. until LOD is less than 5%.
5. Pass granules through Fitzmill @3500 rpm, hammers forward.

### EXAMPLE 2

### TIMERx® Excipient D

TABLE 2

| Ingredients | % |
|---|---|
| Locust Bean Gum | 42 |
| Xanthan Gum | 28 |
| Mannitol | 20 |
| Calcium Sulfate | 10 |
| Total | 100 |

* Purified water used as a processing agent and is removed during drying

The TIMERx® excipient D is prepared by the following steps:
1. Add locust bean gum, xanthan gum, mannitol and calcium sulfate into a high shear granulator.
2. Dry mix material until uniform. 3. Add water (20-50%)to step 2 over a defined time, while mixing at low speed.
4. Granulate at high speed until proper granules form; and optionally
5. Dry in fluid bed dryer.
6. Mill dry material to get proper particle size.

### Preparation of Controlled-Release Compositions

The controlled-release compositions of the present invention are prepared by granulating a controlled-release carrier (Excipients A-d) described above with a emulsion containing a therapeutically effective amount of an active agent as set forth below:

### EXAMPLES 3A-D

### Nimodipine 60 mg Controlled-Release Tablets

Nimodipine has been formulated into an emulsion as follows.
1. Weigh an accurate amount of nimodipine powder
2. Dissolve nimodipine in N-methyl-2-pyrrolidone completely,
3. Add Vitamin E-TPGS to active ingredient solution,
4. Add DI water and shake it until all Vitamin E-TPGS dissolved and a clear transparent solution is formed.

5. Measure emulsion particle size and verify that it is in the range 7.8-20.0 nm. (preferred range is 9.9 to 15.8 nm).

TABLE 3
Nimodipine 60 mg Emulsion Formulation

| | Formulation | | | | | | | |
|---|---|---|---|---|---|---|---|---|
| | Example 3A | | Example 3B | | Example 3C | | Example 3D | |
| | Weight | Weight | Weight | Weight | Weight | Weight | Weight | Weight |
| Component | (grams) | % | (grams) | % | (grams) | % | (grams) | % |
| Nimodipine | 0.06 | 0.55 | 0.06 | 0.60 | 0.06 | 0.60 | 0.06 | 0.52 |
| N-methyl-2-pyrrolidone | 0.4 | 3.65 | 0.6 | 9.94 | 1.0 | 9.94 | 0.36 | 3.15 |
| Vitamin E-TPGS | 2.5 | 22.81 | 0.6 | 9.94 | 3.0 | 29.82 | 3.0 | 26.27 |
| DI Water | 8.0 | 72.99 | 8.0 | 79.52 | 6.0 | 59.64 | 8.0 | 70.05 |
| total | 10.96 | 100 | 10.06 | 100 | 10.06 | 100 | 11.42 | 100 |

These emulsions are tested for stability against 500 times dilution by DI water and different pH medium (pH 1.5, 4.5, 6.8 and 7.5 buffers).

TABLE 4
Phase Behavior of Emulsion against Dilution

| | pH 1.5 | pH 4.5 | pH 6.8 | pH 7.5 |
|---|---|---|---|---|
| Example 3A | Stable clear | Stable clear | Stable clear | Stable clear |
| | solution | solution | solution | solution |
| Example 3B | Stable clear | Stable clear | Stable clear | Stable clear |
| | solution | solution | solution | solution |
| Example 3C | Stable clear | Stable clear | Stable clear | Stable clear |
| | solution | solution | solution | solution |
| Example 3D | Stable clear | Stable clear | Stable clear | Stable clear |
| | solution | solution | solution | solution |

### Tablets Preparation

Nimodipine emulsion controlled-release tablets are prepared as follows:
a) Weigh a portion (4% to 10%, preferred range is 5% to 7.5%) of one of the previously prepared (A-D) emulsions to be used as a wet granulation agent.
b) Premix TIMERx® M70A controlled-release carrier and MCC SP15 for 5 minutes in a planetary, high shear, or fluidized bed granulator.
c) Add the emulsion as wet granulating agent to the controlled release carrier.
d) Dry the wet granules in an air spray dryer, and pass them through a suitable sieve.
e) Blend the resulting granules with the remaining part of pharmaceutically active agent and tablet lubricant.
f) The lubricated granules are compressed into tablets on a Korsh rotary press equipped with 0.374″×0.748″ oval bisect punch.

### EXAMPLES 4-7

TABLE 5
Nimodipine Emulsion Controlled Release Tablets

| | Example 4 | | Example 5 | | Example 6 | | Example 7 | |
|---|---|---|---|---|---|---|---|---|
| | Tablet | weight | Tablet | weight | Tablet | weight | Tablet | weight |
| Ingredient | weight | %/table | weight | %/table | weight | %/table | weight | %/table |
| TIMERx ® M70A | 519.70 | 52.02 | 564.00 | 56.40 | 467.30 | 46.73 | 433.0 | 43.30 |
| *MCC SP15 | 404.30 | 40.47 | 193.30 | 19.33 | 286.00 | 28.60 | 336.4 | 33.64 |
| Vitamin E-TPGS | None | | 137.20 | 13.72 | 136.70 | 13.67 | 119.7 | 11.97 |
| N-methyl-2-pyrrolidone | None | | 28.90 | 2.89 | 30.60 | 3.06 | 28.5 | 28.50 |
| Nimodipine | 59.80 | 5.99 | 60.20 | 6.02 | 63.3 | 6.33 | 66.8 | 6.68 |
| Pruv | 15.20 | 1.52 | 16.30 | 1.63 | 16.1 | 1.61 | 15.4 | 1.54 |
| Total | 998.8 | 100 | 999.9 | 100 | 1000.0 | 100 | 999.9 | 100 |

TIMERx ® M70 A is made by Penwest Pharmaceuticals, Inc., Danbury, CT.
MCC SP15 is silicified microcrystalline cellulose sold under the Trade name of Prosolv ® by JRS Pharma, Patterson, NY.
Example 4 is a control formulation without surfactant and solvent.

### Testing of Tablets

I. Analytical Methods

A. HPLC Assay Method

An Agilent HPLC analysis system equipped with a C₁₈ reverse phase column (4.6×150 mm) is used. An auto-sampler and a pump and a UV detector were used for analysis at 235 nm wavelength. Samples are injected into a 50:20:30 acetonitrile:methanol: KH₂PO₄-H₃PO₄ pH 2.5 buffer mixture used as mobile phase

B. Dissolution Method

A USP type III Vankel dissolution tester was used for all dissolution studies. The dissolution was performed in 250 ml of dissolution medium at a temperature 37° C. with agitation rate of 15 dpm both in pH 1.5 and pH 7.5 solution. Buffer prepared accordingly to USP method. Samples were taken at specific time points and filtered through 1.6 μm filter, and then a portion of them was put into screw cap HPLC glass vials until analysis.

Part of the nimodipine released remained as non-liquid droplets because of non-sink conditions dissolution medium (Sink condition means a 7×amount of dissolution medium than necessary to give a saturated solution at the specified dose, which is not practical, it need about 18 liter dissolution medium for 60 mg nimodipine). Therefore, after finishing dissolution, 25 ml of a 18% (w/v) Cremphor A-25 (BASF Corp.) solution was added to each dissolution vessel to solubilize the remaining nimodipine released. After standing for 24 hours at 30° C., samples are filtered and assayed by HPLC. Post solubilization results obtained were used to calculate the amount of drug released as a function of time.

II. Emulsion System Solubilization Ability in Comparison to IR Product

Solubilization ability of emulsion system was compared to the innovator reference system (Nₘ0: a commercial 30 mg nimodipine capsule sold under the trade name of NIMTOP). Example 6 and Example 7 tablets contains 60 mg active and, to give 60 mg active, two Nₘ0 capsules (30 mg active/per capsule) were separatedly crushed and homogeneized in pH 1.5 and 7.5 buffers and then left at rest for 8 hours. Samples prepared are then filtered and assayed by HPLC to determine the amount of active in the solution.

TABLE 6
Comparison of Dissolved Nimodipine in Controlled
Release Tablets versus Innovator's Capsule
in 250 ml pH 1.5 and 7.5 buffer solutions

| | |
|---|---|
| pH 1.5 | pH 7.5 |

| | | weight % | | weight % |
|---|---|---|---|---|
| Formulation | mg | of dose | mg | of dose |
| Example 6 | 2.45 | 4.08 | 3.32 | 5.53 |
| Example 7 | 6.52 | 9.76 | 5.61 | 8.15 |
| Nₘ0 | 0.39 | 0.65 | 0.20 | 0.33 |

Note:
Nₘ0 is the commercial innovator product, used as reference.

As shown in Table 6, Example 6 and Example 7 emulsions containing tablets give 16 times in pH 1.5 buffer and 28 times in pH 7.5 buffer the solubility of the innovator's capsules.

III. Dissolution Tests Performed at pH 1.5:

TABLE 7
Micrograms of Dissolved Nimodipine from Emulsion
Containing Controlled Release Tablets at Different
Times. (250 ml pH 1.5 buffer)

| Time (hrs) | Example 4 | Example 5 | Example 6 | Example 7 |
|---|---|---|---|---|
| 1.00 | 333.51 | 485.58 | 537.2 | 585.30 |
| 3.00 | 116.39 | 619.34 | 685.38 | 730.71 |
| 6.00 | 282.51 | 625.83 | 685.7 | 906.33 |
| 10.00 | 302.61 | 555.69 | 724.21 | 1220.59 |
| 16.00 | 267.52 | 678.39 | 821.37 | 1475.22 |
| 24.00 | 228.29 | 1120.14 | 1248.86 | 1602.81 |
| Total dissolved(μg) | 1530.83 | 4084.97 | 4702.72 | 6520.96 |
| (% of 60 mg dose) | 2.55% | 6.80% | 7.83% | 11.17% |

TABLE 8
Nimodipine Release from Emulsion Containing Controlled Release
Tablets in 250 mL pH 1.5 buffer (After post dissolution addition
of 25 ml 18% (w/v) Cremphor A-25 solution.)

| Time points | Example 4 | Example 5 | Example 6 | Example 7 |
|---|---|---|---|---|
| (hour) | % rel. | % rel. | % rel. | % rel. |
| 1.00 | 7.19 | 2.4 | 2.16 | 1.97 |
| 3.00 | 10.31 | 7.79 | 4.64 | 5.55 |
| 6.00 | 16.27 | 16.82 | 9.91 | 12.72 |
| 10.00 | 33.27 | 66.87 | 28.78 | 30.75 |
| 16.00 | 57.48 | 91.95 | 50.31 | 58.28 |
| 24.00 | 89.8 | | 87.61 | 84.13 |
| Total released | 89.8 | 91.95 | 87.61 | 84.13 |
| nimodipine (% of | | | | |
| 60 mg dose) | | | | |

Dissolution medium is pH 1.5 potassium chloride/hydrochloric acid buffer.

Tables 7 and 8 show that the amount of nimodipine dissolved from emulsion containing systems is larger (3-4 times) than the control system (no emulsion) even if the initial release rates of emulsion containing systems are lower than control sample. This finding reflects the improvement of nimodipine solubility of TIMERx®/emulsion based tablets. The dissolved amount of nimodipine is less than 100% because of the non-sink n of the dissolution test.

The dissolution data reported in Table 8 shows that the release rate is controlled by of TIMERx® 70A to MCC. Active ingredient can be released in a range of 3 to 24 hours depending on the amount of TIMERx® used.

### Dissolution Tests Performed at pH 7.5

TABLE 9
Micrograms of Dissolved Nimodipine from Emulsion
Containing Controlled Release Tablets at Different
Times. (250 ml pH 7.5 buffer)

| Time (hours) | Example 4 | Example 5 | Example 6 | Example 7 |
|---|---|---|---|---|
| 1 | 494.74 | Awaiting | 391.06 | 416.53 |
| | | results | | |
| 3 | 617.09 | | 490.41 | 686.13 |
| 6 | 635.65 | | 478.46 | 743.68 |
| 10 | 621.65 | | 515.62 | 1038.45 |
| 16 | 594.51 | | 1148.07 | 1481.21 |
| 24 | 548.44 | | 512.38 | 1234.72 |
| Total (μg) | 3512.08 | | 3536 | 5600.72 |
| (% of 60 mg dose) | 5.85% | | 5.89% | 9.59% |

TABLE 10
Nimodipine Release from Emulsion Containing Controlled Release
Tablets in 250 mL pH 7.5 buffer. (After post dissolution addition
of 25 ml 18% (w/v) Cremphor A-25 solution.)

| Time points | | | | |
|---|---|---|---|---|
| (hour) | Example 4 | Example 5 | Example 6 | Example 7 |
| 1.00 | 3.31 | 1.03 | 1.87 | 1.31 |
| 3.00 | 6.31 | 4.11 | 6.01 | 5.91 |
| 6.00 | 16.64 | 11.05 | 20.71 | 17.1 |
| 10.00 | 38.12 | 21.03 | 37.98 | 35.77 |
| 16.00 | 50.06 | 38.24 | 65.92 | 72.4 |
| 24.00 (% of 60 mg dose) | 63.04 | 76.92 | 86.30 | 82.69 |

pH 7.5 buffer made by sodium phosphate monobasic adjusted by sodium hydroxide.

Tables 9 and 10 show that the released rate of nimodipine in 7.5 buffer medium are controlled by the amount of TIMERx® and twice as much nimodipine can be solubilized by emulsion based systems than control sample.

Conclusion:

Data in Tables 6, 7 and 9 show that the amount of nimodipine dissolved from TIMERx® matrix is higher than the control sample and innovator's reference formulation because of the solubilization ability of the emulsion formulation.

Data in Tables 8 and 10 show that release rate of nimodipine is controlled by TIMERx® and that the amount of nimodipine released from the TIMERx® matrix is increased by the presence of a emulsion solubilization system. Therefore, the dissolved amount of nimodipine (which is easy to be absorbed in GI tract) is controlled not only by erosion of TIMERx® gel but also by the solubilization ability of the emulsion system. The release profile of emulsion containing TIMERx® tablets is similar both in pH1.5 and pH 7.5 buffer.

The advantage of emulsion containing formulation is that 60 mg active can be delivered by one 1 g tablet over 3 to 24 hours while to do the same the innovator needs two 1.25 gram capsules and only with immediate release. Our delivery system also exhibits more desirable profile which is insensitive to dissolution medium pH.

### Preparation of Additional Emulsions

### EXAMPLES 8A-D

### Nifedipine 30 mg Emulsions

TABLE 11

| | Formulation | | | | | | | |
|---|---|---|---|---|---|---|---|---|
| | Example 8A | | Example 8B | | Example 8C | | Example 8D | |
| | Weight | Weight | Weight | Weight | Weight | Weight | Weight | Weight |
| Component | (grams) | % | (grams) | % | (grams) | % | (grams) | % |
| Nifedipine | 0.03 | 0.60 | 0.03 | 0.60 | 0.03 | 0.60 | 0.03 | 0.60 |
| N-methyl-2-pyrrolidone | 1.50 | 29.82 | 2.00 | 39.76 | 1.75 | 34.79 | 2.49 | 49.50 |
| Vitamin E-TPGS | 0.8 | 15.90 | 0.4 | 7.95 | 0.4 | 7.95 | 0.4 | 7.95 |
| Lutrol ® F-127 | 0.2 | 3.98 | 0.1 | 1.99 | 0.1 | 1.99 | 0.1 | 1.99 |
| DI water | 2.5 | 49.70 | 2.5 | 49.70 | 2.75 | 54.67 | 2.01 | 39.96 |
| Total | 5.03 | 100 | 5.03 | 100 | 5.03 | 100 | 5.03 | 100 |

*The procedure for making the nifedipine emulsion formulation is the same as for the nimodipine emulsion formulation.

### EXAMPLES 9A-D

### Carvedilol 25 mg Emulsions

TABLE 12

| | Formulation | | | | | | | |
|---|---|---|---|---|---|---|---|---|
| | Example 9A | | Example 9B | | Example 9C | | Example 9D | |
| | Weight | Weight | Weight | Weight | Weight | Weight | Weight | Weight |
| Component | (grams) | % | (grams) | % | (grams) | % | (grams) | % |
| carvedilol | 0.025 | 0.25 | 0.025 | 0.25 | 0.025 | 0.25 | 0.025 | 0.25 |
| Soluphor-P ® | 0.5 | 4.99 | 1.01 | 10.07 | 3.00 | 29.92 | 0.25 | 24.94 |
| Vitamin E-TPGS | 0.33 | 3.29 | 0.66 | 6.58 | 0.66 | 6.58 | 0.84 | 8.38 |
| Lutrol ® F-127 | 0.17 | 1.69 | 0.33 | 3.29 | 0.33 | 3.29 | 0.41 | 4.09 |
| DI water | 9 | 89.77 | 8 | 79.37 | 6 | 59.85 | 8.5 | 84.79 |
| Total | 10.025 | 100 | 10.025 | 100 | 10.025 | 100 | 10.025 | 100 |

*The procedure for making the carvedilol emulsion formulation is the same as for the nimodipine emulsion formulation.

### EXAMPLES 10A-D

### Oxcarbazepine 150 mg Self-Emulsified Formulaiton

TABLE 13

| | Formulation | | | | | | | |
|---|---|---|---|---|---|---|---|---|
| | Example 10A | | Example 10B | | Example 10C | | Example 10D | |
| | Weight | Weight | Weight | Weight | Weight | Weight | Weight | Weight |
| Component | (grams) | % | (grams) | % | (grams) | % | (grams) | % |
| oxcarbazepine | 0.15 | 1.47 | 0.15 | 1.48 | 0.15 | 1.48 | 0.15 | 1.48 |
| N-methyl-2-pyrrolidone | 2.02 | 19.86 | 2.00 | 19.7 | 1.00 | 9.86 | 2.01 | 19.76 |
| Sodium dodecylsulfate | 0.69 | 6.78 | 0.49 | 4.83 | 0.60 | 5.92 | 0.20 | 1.97 |
| PEG-monolaurate | 2.81 | 27.63 | 2.01 | 19.80 | 2.39 | 2.36 | 0.81 | 7.96 |
| DI water | 4.5 | 44.25 | 5.5 | 54.19 | 6.00 | 59.17 | 7.00 | 68.83 |
| Total | 10.17 | 100 | 10.15 | 100 | 10.14 | 100 | 10.17 | 100 |

*The procedure for making the oxcarbazepine emulsion formulation is the same as for the nimodipine emulsion formulation.

### EXAMPLES 11A-D

### Clozapine 25 mg Emulsion Formulation

TABLE 14

| | B | | | | | | | |
|---|---|---|---|---|---|---|---|---|
| | Example 11A | | Example 11B | | Example 11C | | Example 11D | |
| | Weight | Weight | Weight | Weight | Weight | Weight | Weight | Weight |
| Component | (grams) | % | (grams) | % | (grams) | % | (grams) | % |
| clozapine | 0.025 | 2.04 | 0.025 | 1.99 | 0.025 | 1.99 | 0.025 | 1.84 |
| N-methyl-2-pyrrolidone | 0.080 | 6.53 | 0.105 | 8.37 | | | | |
| Soluphor-P ® | | | | | 0.092 | 7.32 | 0.18 | 13.28 |
| V.E-TPGS | | | 0.125 | 9.96 | | | 0.15 | 11.07 |
| Bile extract | 0.12 | 9.79 | | | 0.14 | 11.14 | | |
| DI water | 1.00 | 81.63 | 1.00 | 79.68 | 1.00 | 79.55 | 1.00 | 73.80 |
| Total | 1.225 | 100 | 1.255 | 100 | 1.257 | 100 | 1.355 | 100 |

Procedures to make clozapine-N-methyl-2-pyrrolidone-Vitamin E-TPGS emulsion:
1. Dissolve clozapine in N-methyl-2-pyrrolidone (NMP) solvent, and put this sample into 50° C. oven for 45 minutes. Shake beaker every 10 minutes until all clozapine dissolves and clear brown solution is formed.
2. Melt Vitamin E-TPGS into liquid by using heating plate; control the temperature in the range of 45-50° C. A clear homogeneous solution is formed.
3. Pour the NMP-Clozapine solution made in step 1 into Vitamin FE-TPGS liquid and mix them by stir bar until a clear homogeneous solution is formed. Warm this solution at 40-45° C. to prevent the formation of Vitamin E-TPGS gel.
4. Adding 900 ml DI water into solution made on step 3, accompanying manual stir until clear homogeneous solution is formed. Warm this emulsion by the remaining heat of heating plate and make sure no gel is formed. Otherwise shake the solution until the gel disappears.

### EXAMPLES 12A-B

### pH Dependent Drug Solibilization and Controlled Release Formulation

Clozapine and lot of other drugs present pH dependent solubility behavior. Clozapine has low solubility as pH beyond 5, and fairly good solubility in low pH regions. This could cause high variability in absorption and error absorption in controlled release formulations. In order to overcome the solubility variation, clozapine solubilized by surfactant; solvent and surfactant; organic acid system were developed and incorporated into TIMERx matrix to achieve 24 hour release profile.

### Example 12A

### Solubility of Clozapine in Different pH Medium

TABLE 15

| | DI water | pH 1.5 | pH 7.5 |
|---|---|---|---|
| Solubility (μg/ml) | 11.8 | 712.1 | 30.70 |

### Example 12B

### Selection of Surfactant, Solvents and Organic Acid Used in Clozapine

1. Solubility of Clozapine in Different Surfactants

TABLE 16
Solubility of Clozapine in Surfactant Solution

| Surfactant (1% w/v) | Solubility (μg/ml) |
|---|---|
| PEG-monolaurate | 372.46 |
| Cremphor A25 | 506.16 |
| VE-TPGS | 469.87 |
| Bile extract | 941.15 |
| Cremphor A-6 | 11.5 |
| Cremphor RH 40 | 114.85 |
| PEG distearate | 952.18 |
| Labrasol | 70.64 |
| Lutrol F-68 | 15.3 |
| Sodium desoxycholate | 342.11 |
| Sodium Dodecylate Sulfate (SDS) | 1137.33 |
| PEG 400 | 30.92 |
| PEG 600 | 29.67 |
| Span 20 | 16.14 |
| Tween 80 | 399.19 |
| Tween 20 | 158.44 |
| PEG-diolaurate | 104.48 |
| Solutol HS15 | 344.25 |
| Brij 700 | 336.14 |

Sodium Dodecylate Sulfate, bile extract, PEG distearate show the highest solubilization ability. Considering the anionic polymer nature of TIMERx, compressibility of solid dosage forms, bile extract and Vitamin E-TPGS were chosen as solubilizer.

2. Solubility of Clozapine in Different Solvent

TABLE 17
Solubility of Clozapine in Solvent

| solvent | Solubility (g/ml) |
|---|---|
| N-methyl-2-pyrrolidinone (NMP) | >0.3 g/ml |
| 2-pyrrolidinone (Soluphor-P) | >0.25 g/ml |

Both N-methyl-pyrrolidinone and Soluphor-P have strong solubilization ability.

3. Solubility of Clozapine in Different Organic Acids

TABLE 18
Solubility of Clozapine in Organic Acid and Vitamin
E-TPGS + Organic Acid Combination (Total Concentration
is 1% w/v Except the Given Concentration)

| Acid or acid + surfactant | |
|---|---|
| (1% w/v) | Solubility (μg/ml) |
| Succinic acid (0.05M) | 25730 |
| Ascorbic acid | 258.87 |
| Oleinic acid | 9.86 |
| Alginic acid | 9.99 |
| Stearic acid | Form conjugated compound |
| VE-TPGS + succinic acid* | 19665 |
| VE-TPGS + ascorbic acid* | 10930 |
| VE-TPGS + alginic acid* | 10185 |
| VE-TPGS + stearic acid* | 1730 |
| VE-TPGS + olenic acid* | 5110 |
| Fumaric acid (0.01M) | 8880 |
| Citric acid (0.1M) | 35420 |

*The weight ratio of VE-TPGS to acid was 8:1 and the total solubilizer weight was 72 mg.

The combination of VE-TPGS+succinic acid had the highest solubility and was incorporated into TIMERx matrix.

### EXAMPLES 13A-D

### Surfactant+Solvent Solubilization System

1. Bile Extract+N-methyl-2-Pyrrolidinone (NMP) Solubilization System

Clozapine microemulsion was prepared by weighing the desired amount of clozapine in a vial. N-methyl-2-Pyrrolidinone was then added to the vial and mixed well. Bile extract powder was then added to the mixture of clozapine and N-methyl-2-Pyrrolidinone. Distilled water was utilized to dissolve the bile extract. The vial containing the mixture clozapine, N-methyl-2-Pyrrolidinone and bile extract was shaken until a clear solution was formed.

TABLE 19
50 mg and 25 mg Clozapine Microemulsion Formulation

| | Formulation | | | | | | | |
|---|---|---|---|---|---|---|---|---|
| | A | | B | | C | | D | |
| | Weight | Weight | Weight | Weight | Weight | Weight | Weight | Weight |
| Component | (grams) | % | (grams) | % | (grams) | % | (grams) | % |
| clozapine | 0.05 | 0.50 | 0.025 | 0.48 | 0.05 | 0.51 | 0.05 | 0.50 |
| Bile extract | 0.50 | 4.97 | 0.112 | 2.15 | 0.50 | 4.97 | 4.46 | 44.47 |
| N-methyl-2-Pyrrolidinone | 0.51 | 5.07 | 0.08 | 1.53 | 3.50 | 34.82 | 0.52 | 5.18 |
| DI Water | 9.0 | 89.46 | 5.0 | 95.84 | 6.0 | 59.70 | 5.0 | 49.85 |
| total | 10.06 | 100 | 5.22 | 100 | 10.05 | 100.00 | 10.03 | 100 |

Using formulation B as a solubilization model, the solubilization system was diluted by different buffers. Solubilities of clozapine in different pH are listed in Table 20.

TABLE 20
Solubility of Clozapine-N-methyl-2-Pyrrolidinone-Bile
extract system in different pH's

| medium | Solubility (mg/ml) |
|---|---|
| DI water | 19.81 |
| pH 1.5 buffer | 22.41 |
| pH 6.8 buffer | 8.57 |
| pH 7.5 buffer | 18.15 |

The solubility of clozapine was improved and the solubility variation with pH has was reduced.

2. Bile Extract+2-Pyrrolidinone Solubilization System

TABLE 21
Clozapine (25 mg dose) Solubilized
by Bile Extract and 2-Pyrrolidinone

Formulation

| | Weight | |
|---|---|---|
| Component | (grams) | Weight % |
| clozapine | 0.025 | 0.47 |
| Bile extract | 0.12 | 2.28 |
| 2-Pyrrolidinone | 0.11 | 2.09 |
| DI Water | 5.0 | 95.16 |
| Total | 5.255 | 100 |

TABLE 22
Solubility of Clozapine Solubilized by Bile
Extract/2-Pyrrolidinone in Different Buffers

| medium | Solubility (mg/ml) |
|---|---|
| DI water | 8.8 |
| pH 1.5 buffer | 16.03 |
| pH 4.5 buffer | 13.98 |
| pH 6.8 buffer | 10.89 |
| pH 7.5 buffer | 11.24 |

3. V.E-TPGS+methyl-2-Pyrrolidinone Solubilization System

TABLE 23
Clozapine Solubilized by V.E-TPGS/methyl-2-Pyrrolidinone
System

| | Weight | | Weight | |
|---|---|---|---|---|
| Component | (grams) | Weight % | (grams) | Weight % |
| clozapine | 0.025 | 2.43 | 0.025 | 2.43 |
| VE-TPGS | 0.125 | 12.13 | 0.125 | 12.13 |
| methyl-2-Pyrrolidinone | 0.13 | 12.62 | 0.104 | 10.10 |
| DI Water | 0.75 | 72.82 | 0.775 | 75.25 |
| total | 1.03 | 100 | 1.03 | 100 |

A yellowish clear solution was formed that was stable under 250 times dilution.

TABLE 24
Solubility of Clozapine Solubilized by VE-TPGS/methyl-2-Pyrrolidinone
in Different Buffers

| medium | Solubility (mg/ml) |
|---|---|
| DI water | 13.19 |
| pH 1.5 buffer | 21.01 |
| pH 4.5 buffer | 20.21 |
| pH 6.8 buffer | 17.89 |
| pH 7.5 buffer | 18.02 |

4. VE-TPGS+2-Pyrrolidinone Solubilization System

TABLE 25
Clozapine Solubilized by V.E-TPGS/2-Pyrrolidinone System

| | Weight | | Weight | |
|---|---|---|---|---|
| Component | (grams) | Weight % | (grams) | Weight % |
| clozapine | 0.026 | 1.91 | 0.026 | 1.88 |
| VE-TPGS | 0.15 | 11.03 | 0.15 | 10.87 |
| 2-Pyrrolidinone | 0.18 | 13.23 | 0.20 | 14.49 |
| DI Water | 1 | 73.56 | 1.0 | 72.49 |
| total | 1.36 | 100 | 1.38 | 100 |

TABLE 26
Solubility of Clozapine Solubilized by VE-TPGS/2-Pyrrolidinone
in different buffers

| medium | Solubility (mg/ml) |
|---|---|
| pH 1.5 buffer | 18.69 |
| pH 4.5 buffer | 18.61 |
| pH 6.8 buffer | 18.72 |
| oH 7.5 buffer | 17.93 |

5. VE-TPGS+Organic Acid Solubilization System

TABLE 27
Clozapine Solubilized by VE-TPGS/Succinic Acid System

Formulation

| | Weight | |
|---|---|---|
| Component | (grams) | Weight % |
| clozapine | 0.025 | 2.28 |
| VE-TPGS | 0.064 | 5.83 |
| Succinic acid | 0.008 | 0.73 |
| DI Water | 1.0 | 91.16 |
| total | 1.097 | 100 |

TABLE 28
Solubility of Clozapine/VE-TPGS/Succinic
Acid System in Different Buffers

| Medium | Solubility (mg/ml) |
|---|---|
| DI water | 20.63 |
| pH 1.5 buffer | 21.01 |
| pH 4.5 buffer | 21.24 |
| pH 6.8 buffer | 11.56 |
| pH 7.5 buffer | 22.83 |

### EXAMPLE 14

### Incoporating Solubilization System Into TIMERx Matrix to Achieve 24 Hour Controlled Release Profile

1. Incorporating VE-TPGS+NMP Solubilization System into TIMERx Controlled Release Matrix

Procedure of making clozapine tablet:
a) VE-TPGS, NMP, and clozapine were weighed separately;
b) VE-TPGS was melted completely, the temperature of the water bath was about 50-70° C.;
c) NMP was warmed in a water bath, clozapine was then added into the NMP solvent, the mixture was stirred until the cloazapine completely dissolved. A clear brownish solution formed;
d) A warm solution of clozapine/NMP was added into the VE-TPGS liquid and Mixed completely until a clear homogeneous solution was formed;
e) TIMERx, MCC, Colloidal Silicon Dioxide was premixed in a food processor for about 5 minutes;
f) The solubilized clozapine solution was used as a wet granulation agent; The solubilized clozapine was added into the mixture made in step) to make a wet granulation;
g) Distilled water was used to rinse the container storing the clozapine solution, the rinse was added to the granules to finish the granulation process;
h) The granules were dried for about 30 min at about 38° C. until the Loss on Drying was below 5%;
i) The dried granules were milled through #50 mesh Fiz mill;
j) The milled granules were then V-blended with MCC, CaSO₄, Mg stearate for about 5, 5, and 3 minutes respectively;

k) The mixture was then compressed in a Korsch to make tablets.

TABLE 29
Composition of clozapine solubilized controlled release tablet

| ingredient | mg/tab | % of composition |
|---|---|---|
| clozapine | 25.0 | 4.54 |
| NMP | 64.0 | 11.63 |
| VE-TPGS | 64.0 | 11.63 |
| PEG-4000 | 21.0 | 3.81 |
| MCC (Emcocel 50M) | 70.0 | 12.71 |
| Colloidal Silicon Dioxide (part A) | 40.0 | 7.27 |
| TIMERx M50A | 200.0 | 36.33 |
| CaSO4(compactrol) | 50.0 | 9.08 |
| Colloidal Silicon Dioxide (part B) | 11.0 | 2.00 |
| Mg stearate | 5.5 | 1.00 |
| | 550.5 | 100 |

Part A: inter - granular
Part B: intra - granular

2. Dissolution Result of Clozapine Tablets

Dissolution conditions of pH6.8 buffer (0.1 M Sodium phosphate monobasic) were used as the dissolution medium in all time points;
The pH change was as follows:
   pH 2.5 (0.1N citric acid) in 1st hour; pH 4.5 (potassium phosphate monobasic) in 3rd and 4th hour; pH 6.8 in 8, 16, 24 hour time points;
Dissolution apparatus III with 15 DPM. Time points were: 1, 2, 4, 8, 16, 24 hours;
Clozapine assay method: Agilent HPLC with UV detector was used;
Mobile phase was: Acetonitrile: Methanol: 1.OM Ammonium acetate: DIW 35:40:1:24;
Column was: Inertsil ODS-3 5u, length 50 mm, ID 4.6 mm;

Absorption wavelength was 260 nm. Injection volume: 10 μl, flow rate: 1.0 ml/min.

TABLE 30
Clozapine dissolution in pH 6.8 and pH change

| Time point | Release rate | Release rate |
|---|---|---|
| (hr) | in pH 6.8(%) | in pH change (%) |
| 1 | 14.6 | 33.3 |
| 2 | 19.6 | 40.7 |
| 4 | 29.9 | 53.3 |
| 8 | 50.6 | 72.3 |
| 16 | 80.4 | 97.6 |
| 24 | 98.6 | 107.1 |
| recovery | 100.7 | 107.9 |

TABLE 30
The composition of TIMERx based clozapine reference
formulation (without solubilizer)

| Ingredient | Mg/tab | % |
|---|---|---|
| clozapine | 25 | 24.87 |
| TIMERx M50A | 75 | 74.63 |
| Mg stearate | 0.5 | 0.50 |
| total | 100.5 | 100 |

TABLE 31
Dissolution profile of TIMERx based clozapine reference
(without solubilizer) in pH 6.8 and pH change

| Time point | Release rate | Release rate |
|---|---|---|
| (hr) | in pH 6.8(%) | in pH change (%) |
| 1 | 2.5 | 9.13 |
| 2 | 4.5 | 18.07 |
| 4 | 13.1 | 28.75 |
| 8 | 32.1 | 34.56 |
| 16 | 71.1 | 63.93 |
| 24 | 93.4 | 88.83 |
| recovery | 93.4 | 88.83 |

3. The Effect of Surfactant Level on the Release Profile

Solubilizer with 50 mg and 64 mg levels were incorporated into TIMERx matrix. Their effect on release profile was studied.

TABLE 32
Effect of solubilizer level on release rate

| Ingredients | Mg/tab | Mg/tab |
|---|---|---|
| clozapine | 25 | 25 |
| N-methyl-2-pyrrolidone | 50 | 64 |
| VE-TPGS | 50 | 64 |
| Colloidal Silicon Dioxide | 46 | 46 |
| TIMERx M50A | 200 | 200 |
| MCC (partA) | 100 | 100 |
| MCC (Part B) | 50 | 50 |
| Mg stearate | 3.77 | 5.5 |
| total | 504 | 556 |

TABLE 33
Dissolution results of clozapine tablets
with different solubilizer levels

| | Released rate in | Released rate in |
|---|---|---|
| Time point | pH 6.8 buffer (50 mg | pH 6.8 buffer (64 mg |
| (hr) | solubilizer level) | solubilizer level) |
| 1 | 14.93 | 7.95 |
| 2 | 18.06 | 13.34 |
| 4 | 23.60 | 25.03 |
| 8 | 35.30 | 48.05 |
| 16 | 54.54 | 78.04 |
| 24 | 67.97 | 90.42 |

High level of solubilizer released faster at later stage and had better recovery. The effect of MCC, Colloidal Silicon Dioxide, CaSO₄, Ca(HPO₄), and TIMERx amount on release profile were all evaluated and optimized.

### EXAMPLE 14

### Incorporating VE-TPGS/succinic Acid Solubilization System into TIMERx Matrix

TABLE 34
Composition of clozapine solubilized controlled tablets

| Ingredient | mg/tab | % of composition |
|---|---|---|
| Clozapine | 25.0 | 7.57 |
| Succinic acid | 5.6 | 1.69 |
| VE-TPGS | 44.8 | 13.56 |
| MCC (Emcocel 50M) | 49 | 14.83 |
| Colloidal Silicon Dioxide | 28 | 8.48 |
| TIMERx M50A | 140 | 42.38 |
| CaSO4 | 35 | 10.60 |
| Mg stearate | 2.92 | 0.89 |
| Total | 330.3 | 100.0 |

TABLE 35
Dissolution results of clozapine tablets

| Time point | Release rate | Release rate |
|---|---|---|
| (hr) | in pH 6.8(%) | in pH change (%) |
| 1 | 9.31 | 26.4 |
| 2 | 14.85 | 35.91 |
| 4 | 23.85 | 50.49 |
| 8 | 44.37 | 64.64 |
| 16 | 69.17 | 83.87 |
| 24 | 82.28 | 90.96 |
| Recovery | 87.73 | 93 |

### EXAMPLE 15

### Incorporating Bile Extract/N-methy-2-pyrrolidone Solubilization System Into TIMERx Matrix

TABLE 36
Clozapine solubilized by bile extract/VE-TPGS

| Ingredient | mg/tablet | % of composition |
|---|---|---|
| clozapine | 25 | 3.32 |
| Bile extract | 118.22 | 15.70 |
| NMP | 80.42 | 10.68 |
| TIMERx M50A | 521.84 | 69.31 |
| Mg stearate | 7.45 | 0.99 |
| total | 752.9 | 100 |

TABLE 36
Dissolution results of clozapine tablets

| Time point | Release rate | Release rate |
|---|---|---|
| (hr) | in pH 6.8(%) | in pH change (%) |
| 1 | 6.62 | 0 |
| 2 | 10.6 | 1.3 |
| 4 | 17.36 | 4.69 |
| 8 | 32.63 | 23.4 |
| 16 | 51.45 | 48.12 |
| 24 | 65.95 | 58.74 |

### EXAMPLE 16

Clozaril® (innovator's product), TIMERx based clozapine solubilized formulation (solubilized by VE-TPGS+NMP, and clozapine solubilized by succinic acid), and clozapine TIMERx based reference formulation (without solubilizer) were dosed in 6 beagle dogs. The dose administered was 25 mg. Plasma concentration was analyzed (See: Table 37 below):

TABLE 37
pK of clozapine in different formulations

| | Formulation | | | | | |
|---|---|---|---|---|---|---|
| | T_{lag} | Tₘₐₓ | Cₘₐₓ | | | |
| | (hr) | (hr) | (ng/ml) | AUCₗₐₛₜ | AUC_{inf} | T_{1/2} |
| Clozaril ® | 0.0 | 3.1 | 83.6 | 684.4 | 699.5 | 5.9 |
| Clozapine solubilized | 0.3 | 5.8 | 75.6 | 704.6 | 721.1 | 5.8 |
| by VE-TPGS + N- | | | | | | |
| methyl-2-pyrrolidone | | | | | | |
| Clozapine solubilized | 0.1 | 6.0 | 54.0 | 567.6 | 588.7 | 6.1 |
| by succinic acid | | | | | | |
| Clozapine + TIMERx | 0.5 | 6.8 | 46.5 | 509.3 | 522.1 | 6.2 |
| reference | | | | | | |

### EXAMPLE 17 (Prophetic)

### Transdermal Delivery System with Clozapine+Vitamin E-TPGS/NMP

There is provided a transdermal therapeutic system of the reservoir type. For that purpose, a cover foil of 15 μm thick polyester material is used which may be provided with a skin-coloured coating or may be transparent. The cover foil is heat-moulded onto a laminate that consists of a microporous membrane, a self-adhesive contact adhesive from the group of acrylates, silicones and polyisobutylene with a tackifying resin, and a protective foil. The microporous membrane may be of the MSX 115 4P type and may contain 28% EVA (ethylene vinyl acetate). The protective foil may be a polyester material, siliconised on one side, of 100 μm layer thickness. A cavity is left between the cover foil and the microporous membrane, which is filled with a solubilized material of clozapine and Vitamin E-TPGS and N-methyl-2-pyrrolidone (NMP).

### EXAMPLE 18 (Prophetic)

### Transdermal Delivery System with Nimodipine+Vitamin E-TPGS/NMP

There is provided a transdermal therapeutic system of the reservoir type. For that purpose, a cover foil of 15 μm thick polyester material is used which may be provided with a skin-coloured coating or may be transparent. The cover foil is heat-moulded onto a laminate that consists of a microporous membrane, a self-adhesive contact adhesive from the group of acrylates, silicones and polyisobutylene with a tackifying resin, and a protective foil. The microporous membrane may be of the MSX 115 4P type and may contain 28% EVA (ethylene vinyl acetate). The protective foil may be a polyester material, siliconised on one side, of 100 μm layer thickness. A cavity is left between the cover foil and the microporous membrane, which is filled with a solubilized material of nimodipine and Vitamin E-TPGS and N-methyl-2-pyrrolidone (NMP).

### EXAMPLE 19 (Prophetic)

### Transdermal Delivery System with Clozapine +Vitamin E- TPGS and Organic Acid

There is provided a transdermal therapeutic system of the reservoir type. For that purpose, a cover foil of 15 μm thick polyester material is used which may be provided with a skin-coloured coating or may be transparent. The cover foil is heat-moulded onto a laminate that consists of a microporous membrane, a self-adhesive contact adhesive from the group of acrylates, silicones and polyisobutylene with a tackifying resin, and a protective foil. The microporous membrane may be of the MSX 115 4P type and may contain 28% EVA (ethylene vinyl acetate). The protective foil may be a polyester material, siliconised on one side, of 100 μm layer thickness. A cavity is left between the cover foil and the microporous membrane, which is filled with a solubilized material of clozapine and Vitamin E-TPGS and Succinic acid.

### EXAMPLE 20 (Prophetic)

### Transdermal Delivery System with Nimodipine+Vitamin E-TPGS and Organic Acid

There is provided a transdermal therapeutic system of the reservoir type. For that purpose, a cover foil of 15 μm thick polyester material is used which may be provided with a skin-coloured coating or may be transparent. The cover foil is heat-moulded onto a laminate that consists of a microporous membrane, a self-adhesive contact adhesive from the group of acrylates, silicones and polyisobutylene with a tackifying resin, and a protective foil. The microporous membrane may be of the MSX 115 4P type and may contain 28% EVA (ethylene vinyl acetate). The protective foil may be a polyester material, siliconised on one side, of 100 μm layer thickness. A cavity is left between the cover foil and the microporous membrane, which is filled with a solubilized material of nimodipine and Vitamin E-TPGS and Succinic acid.

In the preceding specification, the invention has been described with reference to specific exemplary embodiments and examples thereof. It will, however, be evident that various modifications and changes may be made thereto without departing from the broader spirit and scope of the invention as set forth in the claims that follow. The specification and drawings are accordingly to be regarded in an illustrative manner rather than a restrictive sense.

What is claimed is:

1. A composition, comprising:
a solubilized material comprising an active agent and at least one oil-based surfactant capable of solubilizing the active agent, the solubilized material dispersed in a controlled-release particulate matrix.

2. A composition comprising:
a solubilized material comprising an active agent and at least one oil-based surfactant capable of solubilizing the active agent, the solubilized material coated onto a at least one pharmaceutically acceptable particulate controlled-release carrier.

3. The composition of claims 1 or 2, wherein a suitable amount of the composition is incorporated into a unit composition.

4. The composition of claim 3, wherein the unit composition is a tablet.

5. The composition of claim 3, wherein the unit composition is a capsule.

6. The composition of claim 1, wherein the controlled-release particulate matrix comprises at least one pharmaceutically acceptable controlled-release carrier.

7. The composition of claims 1 or 2, wherein the active agent is an insoluble active agent having a solubility no greater than 1 part active agent to about 30 to about 100 parts water (sparingly soluble).

8. The composition of claims 1 or 2, wherein the active agent is an insoluble active agent having a solubility no greater than 1 part active agent to about 100 to about 1000 parts water (slightly soluble).

9. The composition of claims 1 or 2, wherein the active agent is an insoluble active agent having a solubility no greater than 1 part active agent to about 1000 to about 10,000 parts water (very slightly soluble).

10. The composition of claims 1 or 2, wherein the active agent is an insoluble active agent having a solubility no greater than 1 part active agent to about 10,000 or more parts water (insoluble).

11. The composition of claims 1 or 2, wherein the active agent is selected from the group consisting of carvedilol, clozapine, nifedipine, nimodipine, oxcarbazepine and carbamazepine.

12. The composition of claims 1 or 2, wherein the oil-based surfactant is a tocopherol, derivative or mixtures thereof.

13. The composition of claim 12, wherein the oil-based surfactant is D-α-tocopherol polyethylene glycol 1000 succinate (Vitamin E TPGS).

14. The composition of claims 1 or 2, wherein the solubilized material further comprises a pharmaceutically acceptable co-solubilizer.

15. The composition of claim 14, wherein the co-solubilizer is selected from the group consisting of ethanol, propylene glycol, transcutol, glycerol, isopranpol, 2-pyrrolidone, N-methyl-2-pyrrolidone, polyethylene glycol, mineral oil, safflower oil, olive oil, coconut oil, sesame oil, corn oil, castor oil, duoprime oil 70, soybean oil, lemon oil, peppermint oil, triacetin, glycofurol, propylene carbonate, dimethyl acetaminde, dimethyl isosorbide, and any combinations or mixtures thereof.

16. The composition of claim 15, wherein the co-solubilizer is N-methyl-2-pyrrolidone (NMP).

17. The composition of claim 15, wherein the co-solubilizer is 2-pyrrolidone.

18. The composition of claims 1 or 2, wherein the solubilized material further comprises a co-surfactant.

19. The composition of claim 18, wherein the co-surfactant is selected from the group consisting of Lutrol®F-127, Lutrole F-88, Brij 700, Cremophor RH40, Cremophor A25, Cremophor A20, Solutol HS-15, polyethyleneglycol distearate and any combinations or mixtures thereof.

20. The composition of claim 19, wherein the ratio of oil-based surfactant or derivative thereof to co-surfactant is from about 1:1 to about 4:1.

21. The composition of claims 2 or 6, wherein the controlled-release carrier is a natural or synthetic gum.

22. The composition of claim 21, wherein the controlled-release carrier is selected from the group consisting of a heteropolysaccharide gum, a homopolysaccharide gum, alginates, gum karaya, pectin, agar, tragacanth, accacia, carrageenan, tragacanth, chitosan, agar, alginic acid, other polysaccharide gums, acacia catechu, salai guggal, indian bodellum, copaiba gum, asafetida, cambi gum, enterolobium cyclocarpum, mastic gum, benzoin gum, sandarac, gambier gum, butea frondosa (Flame of Forest Gum), myrrh, konjak mannan, guar gum, welan gum, gellan gum, tara gum, locust bean gum, carageenan gum, glucomannan, galactan gum, sodium alginate, tragacanth, chitosan, xanthan gum, deacetylated xanthan gum, pectin, sodium polypectate, gluten, karaya gum, tamarind gum, ghatti gum, Accaroid/Yacca/Red gum, dammar gum, juniper gum, ester gum, ipil-ipil seed gum, gum talha (acacia seyal), cultured plant cell gums, modified starch, hydroxypropylmethyl cellulose, hydroxyethyl cellulose, methylcellulose, sodium carboxymethylcellulose, hydroxypropyl cellulose; acrylics, acrylic acid copolymers and mixtures or combinations thereof.

23. The composition of claim 22, wherein the controlled-release carrier is a heteropolysaccharide gum.

24. The composition of claim 23, wherein the controlled-release carrier further comprises a homopolysaccharide gum.

25. The composition of claim 24, wherein the heteropolysaccharide gum is xanthan gum and the homopolysaccharide gum is locust bean gum and the homopolysacccharide gum is capable of cross-linking the heteropolysaccharide gum when exposed to an environmental fluid.

26. The composition of claim 25, wherein the controlled-release carrier further comprises an inert diluent.

27. The composition of claim 26, wherein the inert diluent is selected from the group consisting of a monosaccharide, a disaccharide, a polyhydric alcohol, and mixtures thereof.

28. The composition of claim 27 wherein the inert diluent comprises mannitol.

29. The composition of claim 27, wherein the ratio of the inert diluent to controlled-release carrier is from about 1:5 to about 5:1.

30. The compsition of claim 25, wherein the controlled-release carrier further comprises a hydrophobic material.

31. The composition of claim 30, wherein the hydrophobic material is selected from the group consisting of a hydrophobic polymer, a cellulosic material, an acrylic polymer, a methacrylic acid polymer, a methacrylic copolymer, hydrogenated vegetable oils, zein, an insoluble salt and mixtures thereof.

32. The composition of claim 30, wherein said hydrophobic material comprises ethylcellulose.

33. The composition of claim 25, wherein the controlled-release carrier further comprises an ionizable gel strength enhancing agent capable of crosslinking with the controlled-release carrier and increasing the gel strength when the composition is exposed to an environmental fluid.

34. The composition of claim 33, wherein the ionizable gel strength enhancing agent comprises an alkali metal or an alkaline earth metal sulfate, chloride, borate, bromide, citrate, acetate, or lactate.

35. The composition of claim 34, wherein the ionizable gel strength enhancing agent is selected from the group consisting of calcium sulfate, sodium chloride, potassium sulfate, sodium carbonate, lithium chloride, tripotassium phosphate, sodium borate, potassium bromide, potassium fluoride, sodium bicarbonate, calcium chloride, magnesium chloride, sodium citrate, sodium acetate, calcium lactate, magnesium sulfate, sodium fluoride, and mixtures thereof.

36. The composition of claim 35, wherein the ionizable gel strength enhancing agent comprises calcium sulfate.

37. The composition of claim 26, wherein the inert diluent is from about 1 to about 20% by weight microcrystalline cellulose.

38. The composition of claim 26, wherein the inert diluent comprises from about 1 to about 20% by weight silified microcrystalline cellulose.

39. The composition of claim 4, wherein the tablet further contains a controlled-release coating.

40. The composition of claim 39, wherein the tablet further contains an immediate-release coating comprising additional active agent.

41. The composition of claim 40, wherein the additional active agent is a water soluble or insoluble drug.

42. A method of preparing a pharmaceutical composition comprising:
a) dissolving an active agent in at least one oil-based surfactant capable of solubilizing the active agent;
b) adding an aqueous solution to the active agent/surfactant mixture to form a emulsion:
c) granulating the emulsion with at least one pharmaceutically acceptable controlled-release carrier to form a granulate.

43. The method of claim 42, wherein step a) comprises dissolving an active agent in at least one oil-based surfactant capable of solubilizing the active agent, and a co-solubilizer.

44. The method of claim 43, wherein the active agent is dissolved in the oil-based surfactant prior to addition of the co-solubilizer.

44. The method of claim 43, wherein the active agent is dissolved in the oil-based surfactant together with the co-solubilizer.

45. The method of claim 43, wherein the active agent is dissolved in the co-solubilizer prior to the addition of the oil-based surfactant.

46. The method of claim 42, wherein step a) further comprises the addition of a co-surfactant.

47. The method of claim 42 wherein the active agent is dissolved together with the oil-based surfactant and co-surfactant.

48. The method of claim 42 wherein the active agent is dissolved in the oil-based surfactant prior to addition of the co-surfactant.

49. The method of claim 42, wherein the active agent is dissolved in the co-surfactant prior to the addition of the oil-based surfactant.

50. The method of claims 46, 47, 48 or 49, wherein the co-surfactant is an oil-based surfactant.

51. The method of any of the preceding claims, further comprising the step of incorporating the granulation into an oral solid composition.

52. The method of claim 51, wherein the oral solid composition is a tablet.

53. The method of claim 51, wherein the oral solid composition is a capsule.

54. The method of claim 52, further comprising coating the tablet with a controlled-release coating.

55. The method of claim 52, further comprising coating the tablet with an immediate-release coating.

56. The method of claim 55, wherein the immediate-release coating contains additional active agent.

57. The method of claim 42, wherein the active agent is selected from the group consisting of carvedilol, clozapine, nifedipine, nimodipine, oxcarbazepine, and carbamazepine.

58. The method of claim 42, wherein the oil-based surfactant is a tocopherol, derivative or mixtures thereof.

59. The method of claim 58, wherein the oil-based surfactant is D-α-tocopherol polyethylene glycol 1000 succinate (Vitamin E TPGS).

60. The method of claim 43, wherein the co-solubilizer is selected from the group consisting of is selected from the group consisting of ethanol, propylene glycol, transcutol, glycerol, isopranpol, 2-pyrrolidone, N-methyl-2-pyrrolidone, polyethylene glycol, mineral oil, safflower oil, olive oil, coconut oil, sesame oil, corn oil, castor oil, duoprime oil 70, soybean oil, triacetin, glycofurol, propylene carbonate, dimethyl acetaminde, dimethyl isosorbide, and any combinations or mixtures thereof.

61. The method of claim 60, wherein the co-solubilizer is N-methyl-2-pyrrolidone (NMP).

62. The method of claim 60, wherein the co-solubilizer is 2-pyrrolidone.

63. The method of claim 46, wherein the co-surfactant is selected from the group consisting of Lutrol® F-127, Lutrol® F-88, Brij 700, Cremophor RH40, Cremophor A25, Cremophor A20, Solutol HS-15, polyethyleneglycol distearate and any combinations or mixtures thereof.

64. The method of claim 42, wherein the emulsion is an oil-in-water emulsion.

65. The method of claim 64, wherein the oil-in-water emulsion has a mean droplet size from about 0.01 μm to about 200 μm.

66. The method of claim 46, wherein the ratio of oil-based surfactant or derivative thereof to co-surfactant is from about 1:1 to about 4:1.

67. The method of claim 42, wherein the aqueous solution in step b) is water.

68. The method of claim 42, wherein the controlled-release carrier is a natural or synthetic gum.

69. The method of claim 42, wherein the controlled-release carrier is selected from the group consisting of a heteropolysaccharide gum, a homopolysaccharide gum, alginates, gum karaya, pectin, agar, tragacanth, accacia, carrageenan, tragacanth, chitosan, agar, alginic acid, other polysaccharide gums, acacia catechu, salai guggal, indian bodellum, copaiba gum, asafetida, cambi gum, enterolobium cyclocarpum, mastic gum, benzoin gum, sandarac, gambier gum, butea frondosa (Flame of Forest Gum), myrrh, konjak mannan, guar gum, welan gum, gellan gum, tara gum, locust bean gum, carageenan gum, glucomannan, galactan gum, sodium alginate, tragacanth, chitosan, xanthan gum, deacetylated xanthan gum, pectin, sodium polypectate, gluten, karaya gum, tamarind gum, ghatti gum, Accaroid/Yacca/Red gum, dammar gum, juniper gum, ester gum, ipil-ipil seed gum, gum talha (acacia seyal), cultured plant cell gums, modified starch, hydroxypropylmethyl cellulose, hydroxyethyl cellulose, methylcellulose, sodium carboxymethylcellulose, hydroxypropyl cellulose; acrylics, acrylic acid copolymers and mixtures or combinations thereof.

70. The method of claim 69, wherein the controlled-release carrier is a heteropolysachharide gum.

71. The method of claim 70, wherein the controlled-release carrier further comprises a homopolysaccharide gum.

72. The method of claim 71, wherein the heteropolysaccharide gum is xanthan gum and the homopolysaccharide gum is locust bean gum and the homopolysacccharide gum is capable of cross-linking the heteropolysacchariude gum when exposed to an environmental fluid.

73. The method of claim 42, wherein the controlled-release carrier further comprises an inert diluent.

74. The method of claim 73, wherein the inert diluent is selected from the group consisting of a monosaccharide, a disaccharide, a polyhydric alcohol, and mixtures thereof.

75. The method of claim 73, wherein the inert diluent comprises mannitol.

76. The method of claim 73, wherein the ratio of the inert diluent to controlled-release carrier is from about 1:5 to about 5:1.

77. The method of claim 42, wherein the controlled-release carrier further comprises a hydrophobic material.

78. The method of claim 77, wherein the hydrophobic material is selected from the group consisting of a hydrophobic polymer, a cellulosic material, an acrylic polymer, a methacrylic acid polymer, a methacrylic copolymer, hydrogenated vegetable oils, zein, an insoluble salt and mixtures thereof.

79. The method of claim 77, wherein said hydrophobic material comprises ethylcellulose.

80. The method of claim 42, wherein the controlled-release carrier further comprises an ionizable gel strength enhancing agent capable of crosslinking with the controlled-release carrier and increasing the gel strength when the composition is exposed to an environmental fluid.

81. The method of claim 80, wherein the ionizable gel strength enhancing agent comprises an alkali metal or an alkaline earth metal sulfate, chloride, borate, bromide, citrate, acetate, or lactate.

82. The method of claim 81, wherein the ionizable gel strength enhancing agent is selected from the group consisting of calcium sulfate, sodium chloride, potassium sulfate, sodium carbonate, lithium chloride, tripotassium phosphate, sodium borate, potassium bromide, potassium fluoride, sodium bicarbonate, calcium chloride, magnesium chloride, sodium citrate, sodium acetate, calcium lactate, magnesium sulfate, sodium fluoride, and mixtures thereof.

83. The method of claim 82, wherein the ionizable gel strength enhancing agent comprises calcium sulfate.

84. The method of claim 42, further comprising the step of mixing an inert diluent together with the controlled-release carrier prior to the granulating step (step c)).

85. The method of claim 84, wherein the inert diluent is from about 1 to about 20% by weight microcrystalline cellulose.

86. The method of claim 85, wherein the inert diluent comprises from about 1 to about 20% by weight silicified microcrystalline cellulose.

87. The composition of claim 1, wherein the composition is a transdermal delivery system.

88. The composition of claim 87, wherein the transdermal delivery system is selected from the group consisting of a patch, a cream, a gel, a paste, and a lotion.

89. The composition of claim 87, wherein the transdermal delivery system is a patch.

90. A transdermal delivery system comprising:
a solubilized material comprising an active agent and at least one oil-based surfactant capable of solubilizing the active agent, contained in a patch, a cream, a gel, a paste, and a lotion.

91. The transdermal delivery system of claim 90, wherein the solubilized material is dispersed in a controlled-release carrier.

92. The transdermal delivery system of claim 90, wherein the active agent is an insoluble active agent having a solubility no greater than 1 part active agent to about 30 to about 100 parts water (sparingly soluble).

93. The transdermal delivery system of claim 90, wherein the active agent is an insoluble active agent having a solubility no greater than 1 part active agent to about 100 to about 1000 parts water (slightly soluble).

94. The transdermal delivery system of claim 90, wherein the active agent is an insoluble active agent having a solubility no greater than 1 part active agent to about 1000 to about 10,000 parts water (very slightly soluble).

95. The transdermal delivery system of claim 90, wherein the active agent is an insoluble active agent having a solubility no greater than 1 part active agent to about 10,000 or more parts water (insoluble).

96. The transdermal delivery system of claim 90, wherein the active agent is selected from the group consisting of carvedilol, clozapine, nifedipine, nimodipine, oxcarbazepine and carbamazepine.

97. The transdermal delivery system of claim 90, wherein the oil-based surfactant is a tocopherol, derivative or mixtures thereof.

98. The transdermal delivery system of claim 97, wherein the oil-based surfactant is D-α-tocopherol polyethylene glycol 1000 succinate (Vitamin E TPGS).

99. The transdermal delivery system of claim 90, wherein the solubilized material further comprises a pharmaceutically acceptable co-solubilizer.

100. The transdermal delivery system of claim 99, wherein the co-solubilizer is selected from the group consisting of ethanol, propylene glycol, transcutol, glycerol, isopranpol, 2-pyrrolidone, N-methyl-2-pyrrolidone, polyethylene glycol, mineral oil, safflower oil, olive oil, coconut oil, sesame oil, corn oil, castor oil, duoprime oil 70, soybean oil, lemon oil, peppermint oil, triacetin, glycofurol, propylene carbonate, dimethyl acetaminde, dimethyl isosorbide, and any combinations or mixtures thereof.

101. The transdermal delivery system of claim 100, wherein the co-solubilizer is N-methyl-2-pyrrolidone (NMP).

102. The transdermal delivery system of claim 100, wherein the co-solubilizer is 2-pyrrolidone.

103. The transdermal delivery system of claim 90, wherein the solubilized material further comprises a co-surfactant.

104. The transdermal delivery system of claim 103, wherein the co-surfactant is selected from the group consisting of Lutrol® F-127, Lutrol® F-88, Brij 700, Cremophor RH40, Cremophor A25, Cremophor A20, Solutol HS-15, polyethyleneglycol distearate and any combinations or mixtures thereof.

105. The transdermal delivery system of claim 104, wherein the ratio of oil-based surfactant or derivative thereof to co-surfactant is from about 1:1 to about 4:1.

106. The transdermal delivery system of claims 90-105, wherein the transdermal delivery system is selected from the group consisting of a patch, a cream, a gel, a paste, and a lotion.

107. The transdermal delivery system of claims 90-105, wherein the transdermal delivery system is a patch.

108. The composition of claim 14, wherein the co-solubilizer is an organic acid selected from the group consisting of succinic acid, ascorbic acid, oleinic acid, alginic acid, stearic acid, lenic acid, fumaric acid, and citric acid.

109. The transdermal delivery system of claim 99, wherein the co-solubilizer is an organic acid selected from the group consisting of succinic acid, ascorbic acid, oleinic acid, alginic acid, stearic acid, lenic acid, fumaric acid, and citric acid.
